(19) United States
(12) Patent Application Publication
(10) Pub. No.: US 2018/0181771 A1
(43) Pub. Date: Jun. 28, 2018

(54) INFORMATION PROCESSING APPARATUS, INFORMATION PROCESSING SYSTEM AND INFORMATION PROCESSING METHOD THAT GENERATE CONFIDENTIALIZED PERSONAL INFORMATION

(71) Applicant: FUJITSU LIMITED, Kawasaki-shi (JP)

(72) Inventors: Asuka Okagawa, Kawasaki (JP); Yuri Shimada, Yokohama (JP); Kazunori Kobashi, Yamato (JP); Akimasa Yoshida, Nagoya (JP)

(73) Assignee: FUJITSU LIMITED, Kawasaki-shi (JP)

(21) Appl. No.: 15/818,845

(22) Filed: Nov. 21, 2017

(30) Foreign Application Priority Data
Dec. 28, 2016 (JP) 2016-256815

(51) Int. Cl. G06F 21/62

(52) U.S. Cl. CPC G06F 21/6245; G16H 10/60

(57) ABSTRACT

A memory stores first confidentialization-level information, which represents a confidentialization level of a first confidentialization process. A processor generates first confidentialized personal information by applying the first confidentialization process to personal information provided from an information provision institution. A communication interface circuit transfers the first confidentialized personal information to a storage device used by an information analysis institution. Next, the processor compares the first confidentialization-level information and second confidentialization-level information, which represents a confidentialization level requested by the information analysis institution for a second confidentialization process, and generates a comparison result. Then, the processor generates second confidentialized personal information by applying the second confidentialization process to the personal information on the basis of the comparison result, and the communication interface circuit transfers the second confidentialized personal information to the storage device.

### CROSS-REFERENCE TO RELATED APPLICATION

This application is based upon and claims the benefit of priority of the prior Japanese Patent Application No. 2016-256815, filed on Dec. 28, 2016, the entire contents of which are incorporated herein by reference.

### FIELD

The embodiments discussed herein are related to an information processing apparatus, an information processing system and an information processing method.

### BACKGROUND

In recent years, there has been an increasing demand for big data analysis. In order to obtain more accurate and more useful analysis results in big data analysis, it is desirable to collect as many data samples as possible.

The government of Japan has a plan to carry out policies to promote big data analysis in the domestic medical field in the future. This plan aims at a situation where pieces of data of electronic medical records are collected from hospitals, the collected pieces of data are processed into anonymous data, and groups that wish to use the anonymous data are provided with the data as data available for big data analysis.

Electronic medical records are data including much personal information that is related to the privacy of patients. Thus, it is desirable that measures be taken to prevent leaks of personal information when a great amount of this kind of data is collected.

Techniques for utilizing medical record information of a patient, medical information obtained from a patient or a sample, or other information are also known (see for example Patent Documents 1 and 2).

Patent Document 1: International Publication Pamphlet No. WO 2003/030047

Patent Document 2: Japanese Laid-open Patent Publication No. 2005-293273

### SUMMARY

According to an aspect of the embodiments, an information processing apparatus includes a memory, a processor coupled to the memory, and a communication interface circuit. The memory stores first confidentialization-level information, which represents a confidentialization level of a first confidentialization process, and the processor generates first confidentialized personal information by applying the first confidentialization process to personal information provided from an information provision institution. The communication interface circuit transfers the first confidentialized personal information to a storage device used by an information analysis institution.

Next, the processor compares the first confidentialization-level information and second confidentialization-level information, which represents a confidentialization level requested by the information analysis institution for a second confidentialization process, and generates a comparison result. Then, the processor generates second confidentialized personal information by applying the second confidentialization process to the personal information provided from the information provision institution on the basis of the comparison result, and the communication interface circuit transfers the second confidentialized personal information to the storage device.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a configuration diagram of the information processing system of a prior application;

FIG. 2 is a configuration diagram of an information processing system according to an embodiment;

FIG. 3 is a flowchart of a confidentialization process;

FIG. 4 illustrates a specific example of an information processing system;

FIG. 5 is a configuration diagram of a hospital system;

FIG. 6 is a configuration diagram of a backup storage device;

FIG. 7 is a functional configuration diagram of a VM;

FIG. 8 is a configuration diagram of a collection storage device;

FIG. 9 illustrates a basic table included in personal information;

FIG. 10 illustrates a consultation table included in personal information;

FIG. 11 illustrates confidentialization level information used in mode M1;

FIG. 12 illustrates confidentialization level information used in mode M2;

FIG. 13 illustrates an ID table;

FIG. 14 illustrates a time-date table;

FIG. 15 illustrates a process table;

FIG. 16 illustrates a basic table included in confidentialized personal information;

FIG. 17 illustrates a consultation table included in confidentialized personal information;

FIG. 18 illustrates a process of converting a data format;

FIG. 19 illustrates an information provision sequence in mode M1;

FIG. 20A is a diagram illustrating an operation sequence in mode M2 (first part);

FIG. 20B is a diagram illustrating an operation sequence in mode M2 (second part);

FIG. 20C is a diagram illustrating an operation sequence in mode M2 (third part);

FIG. 20D is a diagram illustrating an operation sequence in mode M2 (fourth part);

FIG. 20E is a diagram illustrating an operation sequence in mode M2 (fifth part);

FIG. 20F is a diagram illustrating an operation sequence in mode M2 (sixth part);

FIG. 20G is a diagram illustrating an operation sequence in mode M2 (seventh part);

FIG. 20H is a diagram illustrating an operation sequence in mode M2 (eighth part);

FIG. 20I is a diagram illustrating an operation sequence in mode M2 (ninth part);

FIG. 20J is a diagram illustrating an operation sequence in mode M2 (tenth part);

FIG. 20K is a diagram illustrating an operation sequence in mode M2 (eleventh part);

FIG. 20L is a diagram illustrating an operation sequence in mode M2 (twelfth part); and

FIG. 21 is a hardware configuration diagram of an information processing apparatus.

### DESCRIPTION OF EMBODIMENTS

Hereinafter, the embodiments will be explained in detail by referring to the drawings.

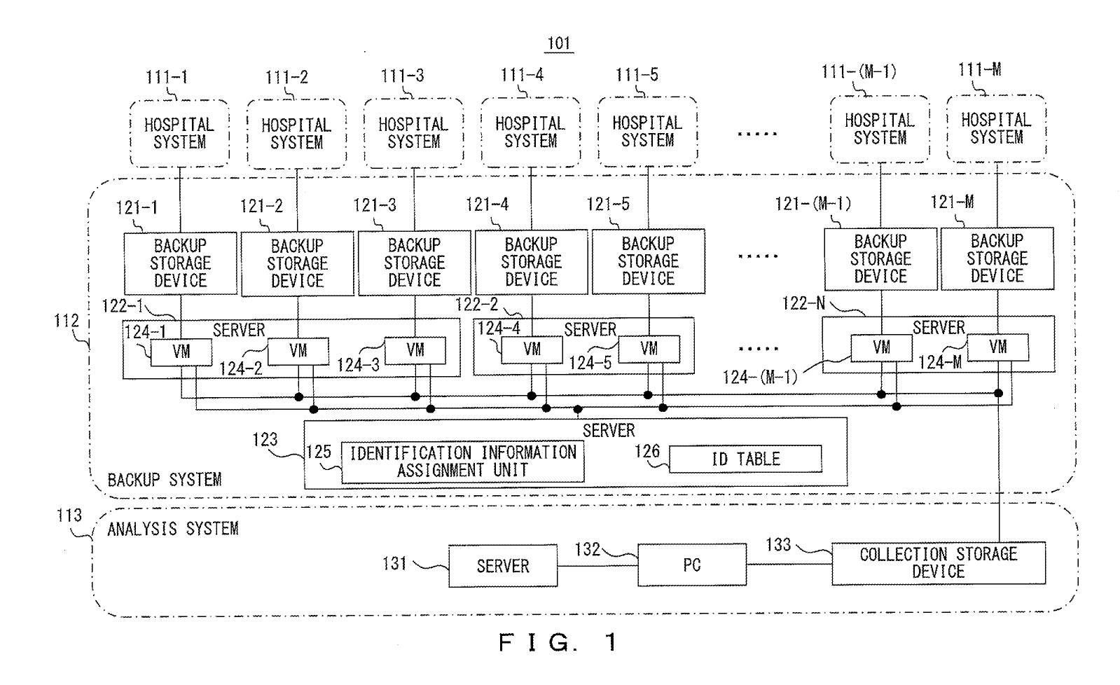

FIG. 1 illustrates a configuration example of an information processing system described in Japanese Patent Application No. 2016-213590, which is a prior application. In the information processing system illustrated in FIG. 1, an information provision institution is a hospital that provides data of electronic medical records, and an information analysis institution is an institution such as the government etc. that collects and analyzes data of electronic medical records.

An information processing system 101 illustrated in FIG. 1 includes hospital systems 111-1 through 111-M (M is an integer that is equal to or greater than 2), a backup system 112 and an analysis system 113. The hospital system 111-i (i=1 through M) is the hospital system of the i-th hospital.

The backup system 112 includes backup storage devices 121-1 through 121-M, servers 122-1 through 122-N(N is an integer that is equal to or greater than 1 and equal to or smaller than M) and a server 123.

In each server 122-j (j=1 through N), a virtual machine (VM) of at least one hospital operates. In this example, a VM 124-1 of the first hospital, a VM 124-2 of the second hospital and a VM 124-3 of the third hospital are operating in the server 122-1. The VM 124-1 of the fourth hospital and the VM 124-3 of the fifth hospital are operating in the server 122-2, and a VM 124-(M−1) of the (M−1)-th hospital and a VM 124-M of the M-th hospital are operating in the server 122-N.

The server 123 includes an identification information assignment unit 125 and stores an ID table 126. The ID table 126 includes a correspondence relationship for associating personal identification information (personal ID) included in an electronic medical record and common identification information (common ID) for identifying the person across the M hospitals in a shared manner.

The analysis system 113 includes a server 131, a personal computer (PC) 132 and a collection storage device 133.

The information processing system 101 illustrated in FIG. 1 can operate in mode M1, in which a confidentialization process is performed on the basis of a request from each hospital, and mode M2, in which a confidentialization process is performed on the basis of a request from an information analysis institution. When the information processing system 101 operates in mode M1, an electronic medical record is analyzed in for example the following procedures.

(P11) A clerk or a patient of each hospital inputs confidentialization level information, which represents the confidentialization level desired by the patient, for each item included in an electronic medical record of the hospital system 111-i. The confidentialization level of each item is represented by for example one of the symbols of “∘”, “Δ” and “x”. “∘” represents information that can be provided without being confidentialized, “Δ” represents information that can be provided when it is processed so that the individual person is not identified, and “x” represents information that is not provided at all. Items for which “Δ” or “x” is set are targets of a confidentialization process.

(P12) The hospital system 111-i stores the input confidentialization level information.

(P13) A doctor of each hospital inputs consultation information of the patient to the electronic medical record.

(P14) The hospital system 111-i stores the input consultation information as personal information of the patient.

(P15) A system administrator of each hospital periodically makes backups. Then, the hospital system 111-i transfers copies of the personal information and the confidentialization level information to the backup storage device 121-i. The backup storage device 121-i stores the copies of the personal information and the confidentialization level information.

(P16) The hospital system 111-i periodically transmits a confidentialization request to the VM 124-i, and the VM 124-i sets, on the basis of the confidentialization request, a confidentialization target time and date, which represents a time range in which the personal information is a target of the confidentialization process.

(P17) The VM 124-i refers to the confidentialization target time and date and a confidentialization completion time and date, which represents the progress of the confidentialization process, and determines whether or not to perform the confidentialization process.

(P18) When the confidentialization process is to be performed, the VM 124-i searches the personal information in the backup storage device 121-i for an entry whose time and date of updating is later than the confidentialization completion time and date.

(P19) The VM 124-i converts the data formats of respective entries of the personal information into a uniform data format by using a conversion program of each hospital.

(P20) The identification information assignment unit 125 of the server 123 refers to the ID table 126 and assigns a common ID corresponding to the personal ID included in each entry in the personal information to that entry.

(P21) The VM 124-i refers to the confidentialization level information of the patient corresponding to each entry, confidentializes the information of an item that is a confidentialization target, and generates confidentialized personal information. Then, the hospital ID is assigned to each entry of the confidentialized personal information. For example, the information of an item for which “∘” is set is not converted, and the information of an item for which “Δ” is set is converted into simplified information by using a prescribed process table. Also, information of an item for which “x” is set is converted into data indicating that the information of the item has been confidentialized.

(P22) The VM 124-i transfers the confidentialized personal information to the collection storage device 133, and the collection storage device 133 stores the confidentialized personal information.

(P23) An analyst of an information analysis institution uses the PC 132 to analyze the confidentialized personal information and stores the analysis result in the server 131. The analysis result is provided to an information user such as a research institution, a pharmaceutical company, etc.

When the information processing system 101 illustrated in FIG. 1 operates in mode M2, an electronic medical record is analyzed in for example the following procedures.

(P31) The hospital system 111-i performs operations that are similar to those of (P11) through (P15) in mode M1.

(P32) An analyst of an information analysis institution uses the PC 132 to transmit, to the VM 124-i, an information provision request together with the process table and confidentialization level information specified by the information analysis institution.

(P33) The VM 124-i switches the process table that it refers to in a confidentialization process from a prescribed process table to the process table specified by the information analysis institution.

(P34) The VM 124-i switches the confidentialization level information that it refers to in a confidentialization process from the confidentialization level information in the backup storage device 121-i to the confidentialization level information specified by the information analysis institution.

(P35) The VM 124-i sets a confidentialization completion time and date and a confidentialization target time and date on the basis of a collection period specified by the information provision request.

(P36) The VM 124-i searches the personal information in the backup storage device 121-i for an entry whose time and date of updating is later than the confidentialization completion time and date.

(P37) The VM 124-i converts the data formats of respective entries of the personal information into a uniform data format by using a conversion program of each hospital.

(P38) The identification information assignment unit 125 of the server 123 refers to the ID table 126 and assigns a common ID corresponding to the personal ID included in each entry in the personal information to that entry.

(P39) The VM 124-i refers to the confidentialization level information specified by the information analysis institution, confidentializes the information of an item that is a confidentialization target, and generates confidentialized personal information. Then, the hospital ID is assigned to each entry of the confidentialized personal information.

(P40) The VM 124-i transfers the confidentialized personal information to the collection storage device 133, and the collection storage device 133 stores the confidentialized personal information.

(P41) An analyst of an information analysis institution uses the PC 132 to analyze the confidentialized personal information, and stores the analysis result in the server 131.

In the case of mode M2, the VM 124-i through the VM 124-M of a plurality of hospitals simultaneously operate and simultaneously transfer confidentialized personal information to the collection storage device 133, which increases the loads on the communication network between the backup system 112 and the analysis system 113. In view of this, it may be possible to reuse confidentialized personal information that has already been stored in the collection storage device 133, for a period that is a target of a confidentialization process in mode M1 and that is included in a collection period specified by an information provision request.

In such a case, it is desirable to again confidentialize, in the VM 124-i, an item to which a confidentialization process not based on the confidentialization level requested by the information analysis institution has been applied in confidentialized personal information generated in mode M1. Thereby, the collection storage device 133 can overwrite and modify an item corresponding to confidentialized personal information that has already been stored.

However, even when confidentialized personal information generated in mode M1 is checked, it is not known whether or not a confidentialization level corresponding to each item is equal to the confidentialization level requested by an information analysis institution. For example, an item that has been converted into data indicating that the information has been confidentialized can be determined to have “x” as the confidentialization level, whereas it is difficult to determine which of “∘” and “Δ” other items have.

Note that this problem arises not only in a case when electronic medical records are collected in hospitals but also in a case when pieces of other types of personal information are collected in other types of information provision institutions.

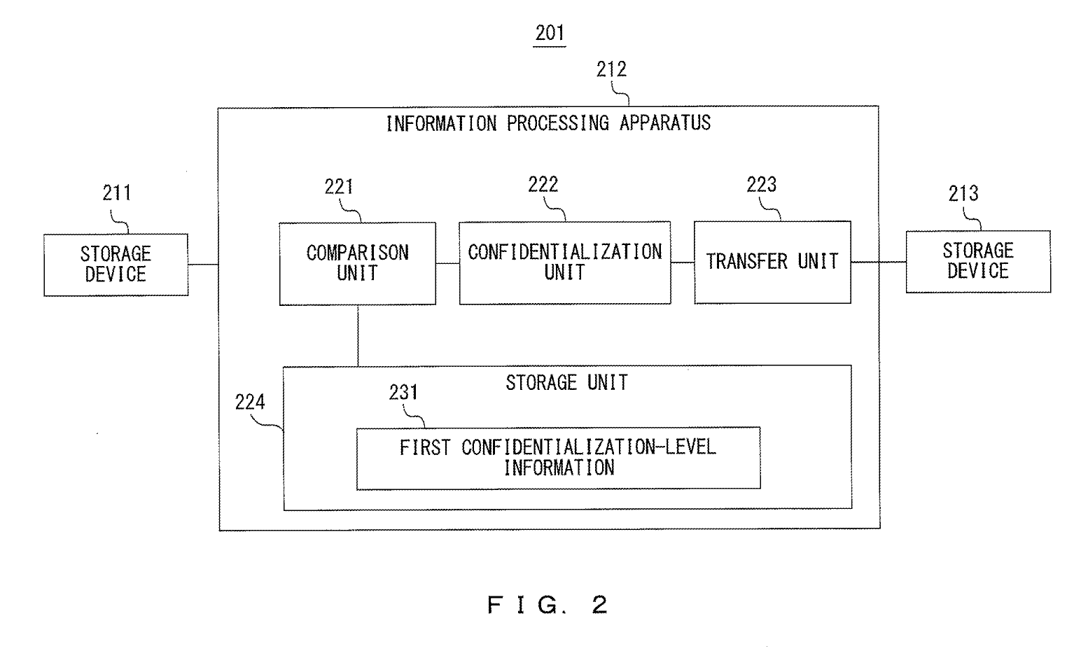

FIG. 2 illustrates a configuration example of an information processing system according to an embodiment. An information processing system 201 illustrated in FIG. 2 includes a storage device 211, an information processing apparatus 212 (computer) and a storage device 213, and the information processing apparatus 212 includes a comparison unit 221, a confidentialization unit 222, a transfer unit 223 and a storage unit 224. The storage device 211 stores personal information provided from an information provision institution, and the storage device 213 is used by an information analysis institution.

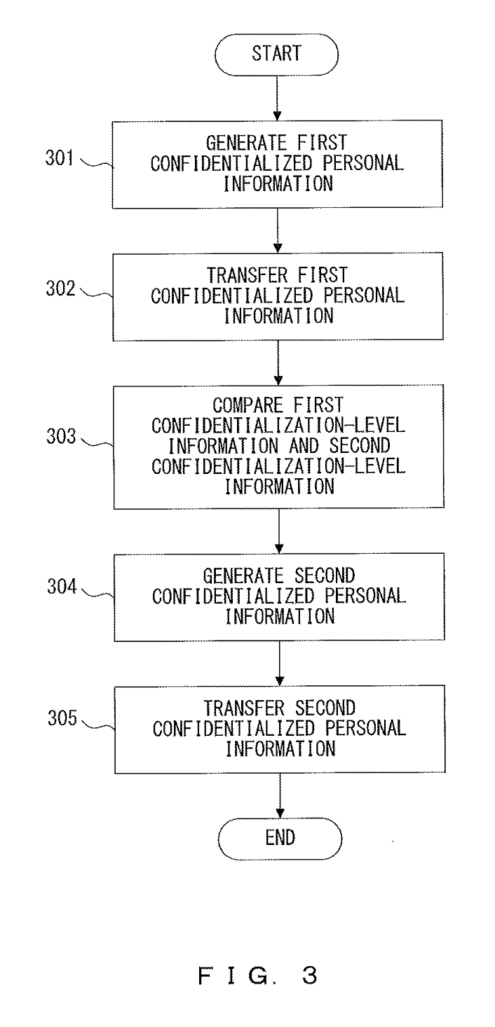

FIG. 3 is a flowchart illustrating an example of a confidentialization process performed by the information processing apparatus 212 illustrated in FIG. 2. First, the confidentialization unit 222 applies a first confidentialization process to personal information stored in the storage device 211, and thereby generates first confidentialized personal information, and the storage unit 224 stores first confidentialization-level information 231, which represents the confidentialization level of the first confidentialization process (step 301). Then, the transfer unit 223 transfers the first confidentialized personal information to the storage device 213 (step 302).

Next, the comparison unit 221 compares the first confidentialization-level information 231 and second confidentialization-level information, which represents the confidentialization level of the second confidentialization process requested by the information analysis institution, and generates a comparison result (step 303). The confidentialization unit 222 applies the second confidentialization process to the personal information on the basis of the comparison result, and thereby generates second confidentialized personal information (step 304), and the transfer unit 223 transfers the second confidentialized personal information to the storage device 213 (step 305).

The information processing system 201 as described above makes it possible to provide confidentialized personal information corresponding to the confidentialization level requested by an information analysis institution.

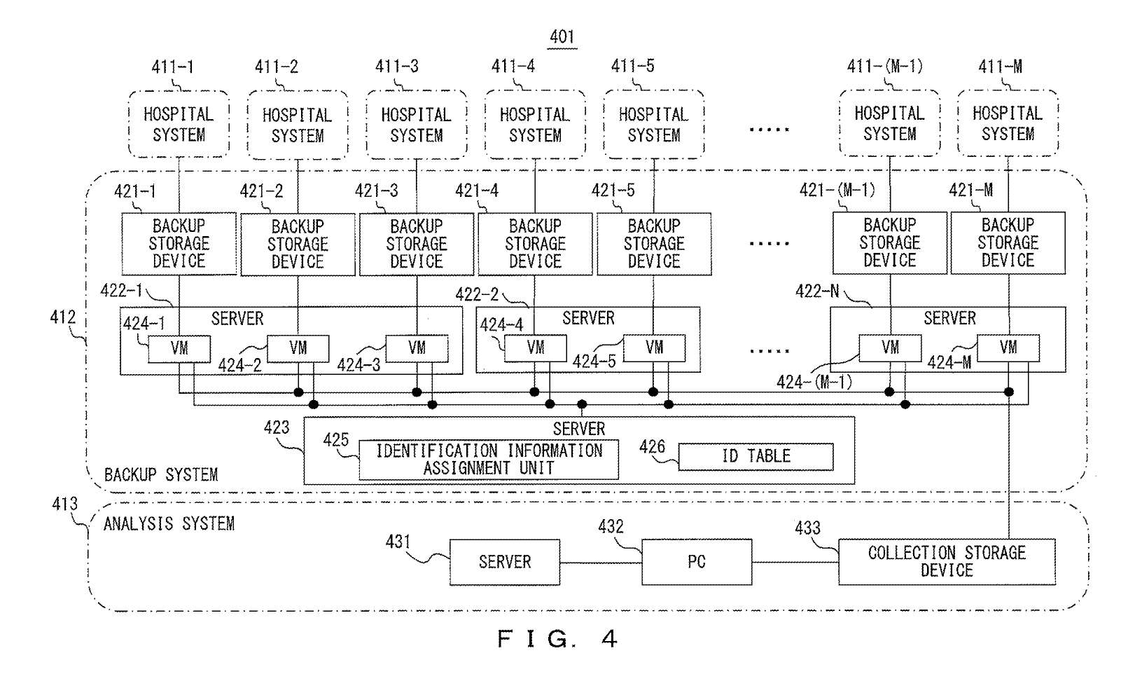

FIG. 4 illustrates a specific example of the information processing system 201 illustrated in FIG. 2. An information processing system 401 illustrated in FIG. 4 includes hospital systems 411-1 through 411-M (M is an integer that is equal to or greater than 2), a backup system 412 and an analysis system 413. The hospital system 411-i (i=1 through M) is the hospital system of the i-th hospital. The M hospitals may be for example hospitals located across the nation or may be hospitals that are located in a specific region.

The backup system 412 is provided in for example a backup site in a communication network such as the Internet etc. and includes the backup storage device 421-1 through the backup storage device 421-M. The backup system 412 further includes servers 422-1 through 422-N(N is an integer that is equal to or greater than 1 and equal to or smaller than M) and a server 423.

In each server 422-j (j=1 through N), a VM of at least one hospital operates. In this example, a VM 424-1 of the first hospital, a VM 424-2 of the second hospital and a VM 424-3 of the third hospital are operating in the server 422-1. The VM 424-1 of the fourth hospital and the VM 424-3 of the fifth hospital are operating in the server 422-2, and a VM 424-(M−1) of the (M−1)-th hospital and a VM 424-M of the M-th hospital are operating in the server 422-N.

Server 423 includes an identification information assignment unit 425 and stores an ID table 426. The ID table 426 includes a correspondence relationship for associating a personal ID included in an electronic medical record and a common ID for identifying the person across the M hospitals in a shared manner.

The analysis system 413 includes a server 431, a PC 432 and a collection storage device 433. Scale-out may be performed for the collection storage device 433 with an increase in the number of hospitals.

The backup storage device 421-1 through the backup storage device 421-M correspond to the storage device 211 illustrated in FIG. 2, and the server 422-1 through the server 422-N correspond to the information processing apparatus 212. Also, the collection storage device 433 corresponds to the storage device 213.

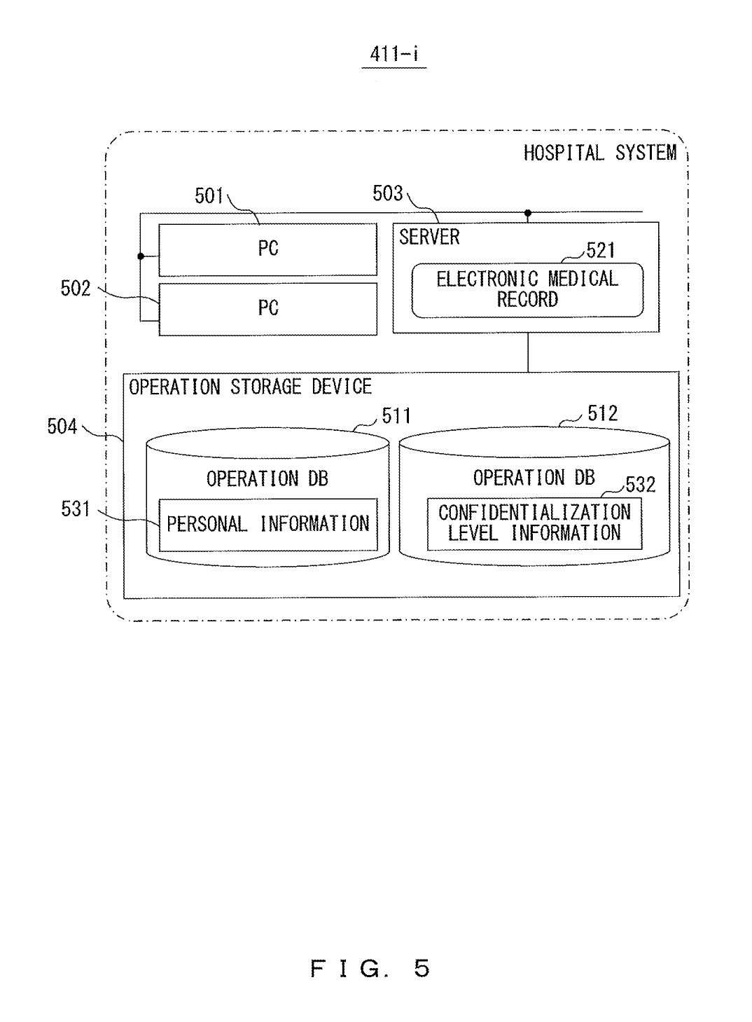

FIG. 5 illustrates a configuration example of the hospital system 411-i illustrated in FIG. 4. The hospital system 411-i illustrated in FIG. 5 includes a PC 501 of a clerk, a PC 502 of a doctor, a server 503 and an operation storage device 504 of each hospital. The PC 501, the PC 502, the server 503 and the operation storage device 504 are connected via for example a Local Area Network (LAN).

The server 503 stores an electronic medical record 521. The operation storage device 504 includes an operation DB 511 and an operation DB 512. The operation DB 511 stores personal information 531, and the operation DB 512 stores confidentialization level information 532.

The personal information 531 is consultation information of a patient recorded in the electronic medical record 521, and the confidentialization level information 532 is information representing the confidentialization level of each of a plurality of items included in the personal information 531. The confidentialization level of each item is specified by for example the patient himself or herself and is applied to the personal information 531 of that patient.

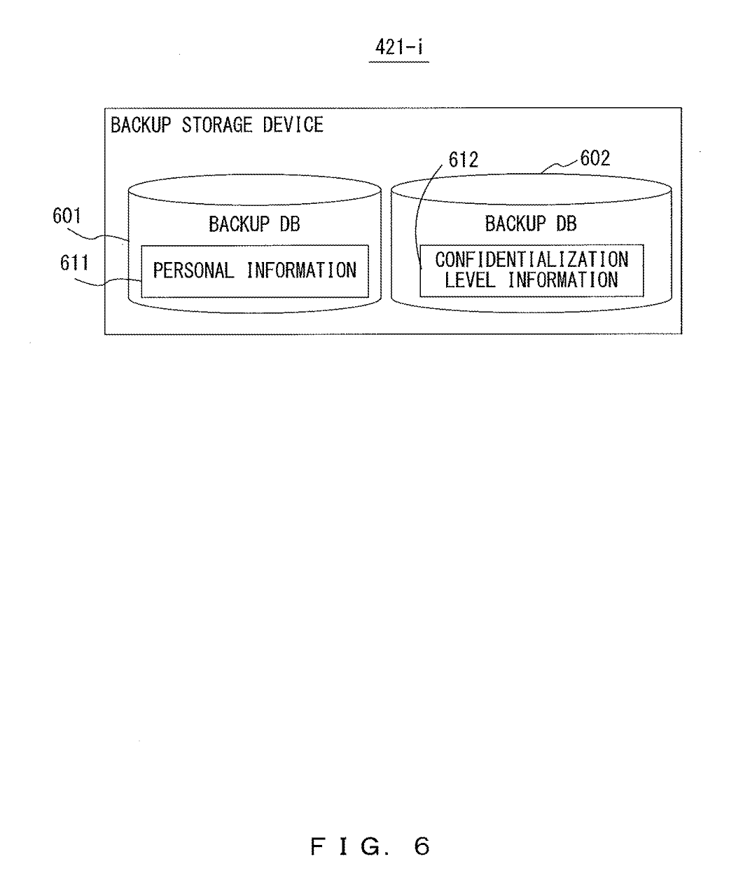

FIG. 6 illustrates a configuration example of the backup storage device 421-i illustrated in FIG. 4. The backup storage device 421-i illustrated in FIG. 6 includes a backup database (DB) 601 and a backup DB 602. The backup DB 601 stores personal information 611, and the backup DB 602 stores confidentialization level information 612. The personal information 611 and the confidentialization level information 612 are respectively copies of the personal information 531 and the confidentialization level information 532 illustrated in FIG. 5.

The ID table 426 illustrated in FIG. 4 includes a correspondence relationship for associating a personal ID included in the personal information 611 and a common ID. The identification information assignment unit 425 refers to the ID table 426 and assigns a common ID corresponding to a personal ID included in the personal information 611 to the personal information 611.

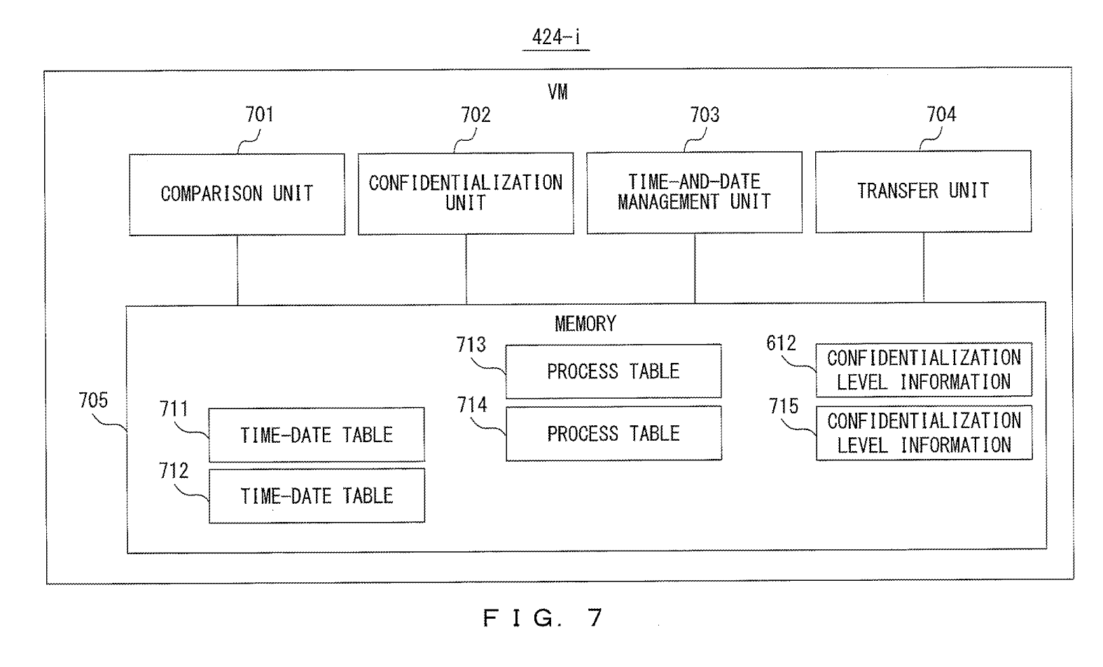

FIG. 7 illustrates a functional configuration example of the VM 424-i illustrated in FIG. 4. The VM 424-i illustrated in FIG. 7 includes a comparison unit 701, a confidentialization unit 702, a time-and-date management unit 703, a transfer unit 704 and a memory 705. The comparison unit 701, the confidentialization unit 702, the time-and-date management unit 703 and the transfer unit 704 are applications executed by the VM 424-i. The comparison unit 701, the confidentialization unit 702 and the transfer unit 704 respectively provide functions similar to those provided by the comparison unit 221, the confidentialization unit 222 and the transfer unit 223 illustrated in FIG. 2.

The memory 705 corresponds to a storage area in the storage unit of the server 422-j and stores the confidentialization level information 612, the time-date table 711, the time-date table 712, the process table 713, the process table 714 and the confidentialization level information 715. The memory 705 corresponds to the storage unit 224 illustrated in FIG. 2, the confidentialization level information 612 corresponds to the first confidentialization-level information 231, and the confidentialization level information 715 corresponds to the second confidentialization-level information.

The time-date table 711 and the time-date table 712 include the target time and date and the completion time and date of a confidentialization process for the personal information 611 of the i-th hospital. The process table 713 and the process table 714 are tables for converting the information of a specific item included in the personal information 611 into simplified information and include a correspondence relationship for associating information before the conversion and the information after the conversion.

The confidentialization level information 715 is information representing the confidentialization level of each of a plurality of items included in the personal information 611. The confidentialization level of each item is specified by for example an institution such as the government, which is not a patient.

The comparison unit 701 compares the confidentialization level information 612 illustrated in FIG. and the confidentialization level information 715 and generates a comparison result. In accordance with the confidentialization level information 612 or the confidentialization level information 715, the confidentialization unit 702 confidentializes the personal information 611 to which a common ID has been assigned and generates confidentialized personal information. The time-and-date management unit 703 updates entries in the time-date table 711 and the time-date table 712, and the transfer unit 704 transfers the confidentialized personal information to the collection storage device 433.

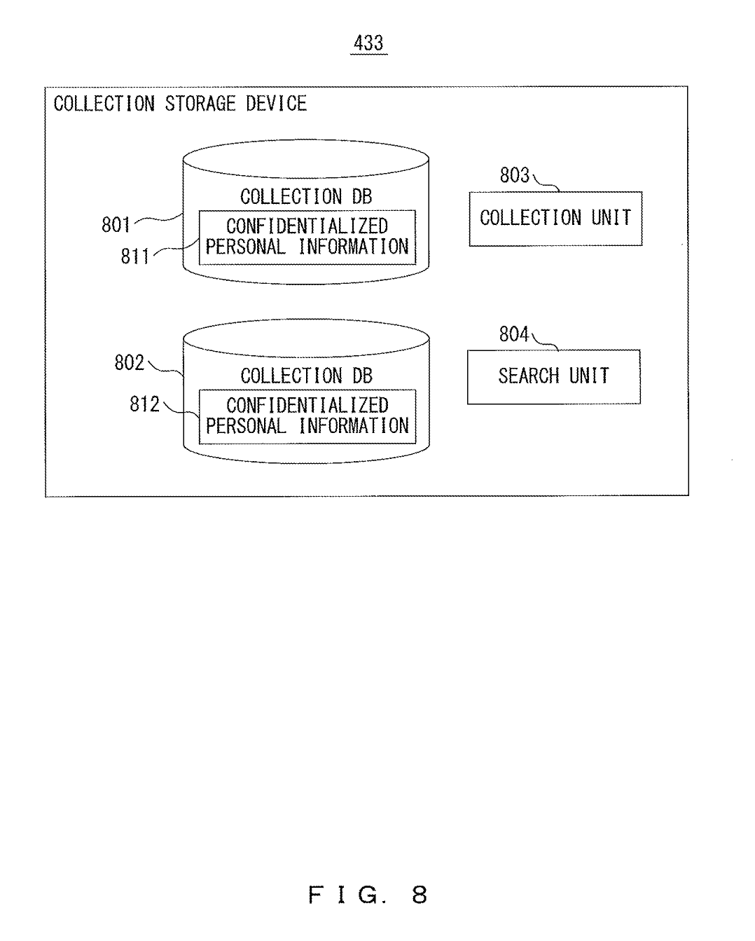

FIG. 8 illustrates a configuration example of the collection storage device 433 illustrated in FIG. 4. The collection storage device 433 includes a collection DB 801, a collection DB 802, a collection unit 803 and a search unit 804. The collection DB 801 stores confidentialized personal information 811 generated by the confidentialization level information 612, and the collection DB 802 stores the confidentialized personal information 812 generated in accordance with the confidentialization level information 715.

The collection unit 803 instructs the search unit 804 to make a copy of the confidentialized personal information 811 that is in the collection DB 801. Then, the search unit 804 searches the confidentialized personal information 811 for an entry that is within a period overlapping a collection period specified by an information analysis institution and stores a copy of the entry for which the search was conducted.

Similarly to the information processing system 101 illustrated in FIG. 1, the information processing system 401 illustrated in FIG. 4 can operate in mode M1, in which a confidentialization process is performed on the basis of a request from each hospital, and mode M2, in which a confidentialization process is performed on the basis of a request from an information analysis institution.

In mode M1, the VM 424-i performs a confidentialization process on the personal information 611 by using the time-date table 711, the process table 713 and the confidentialization level information 612. In mode M2, the VM 424-i performs a confidentialization process on the personal information 611 by using the time-date table 712, the process table 714 and the confidentialization level information 715.

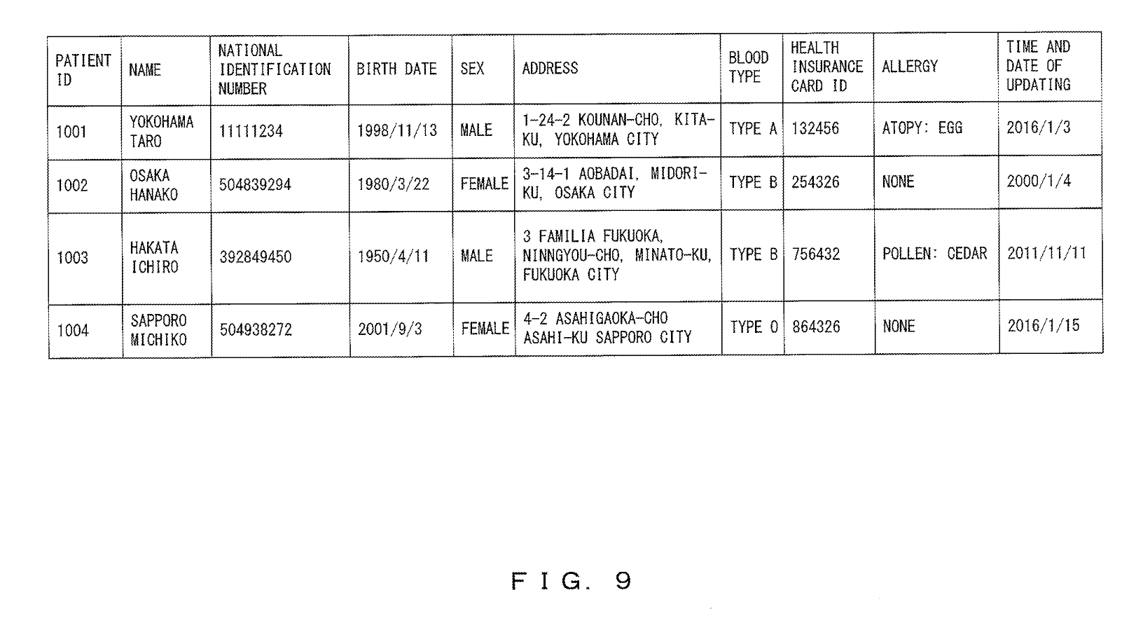

FIG. 9 illustrates an example of a basic table included in the personal information 531 and the personal information 611. The basic table illustrated in FIG. 9 is a table in which basic information of a patient is registered and includes items of patient ID, name, national identification number, birth date, sex, address, blood type, health insurance card ID, allergy and time and date of updating. A patient ID is an ID assigned to a patient by each hospital, a national identification number is an ID assigned to citizens by the government, and a health insurance card ID is an ID assigned to an insured person by an insurer. A time and date of updating represents a time and date at which the basic information of each patient was updated.

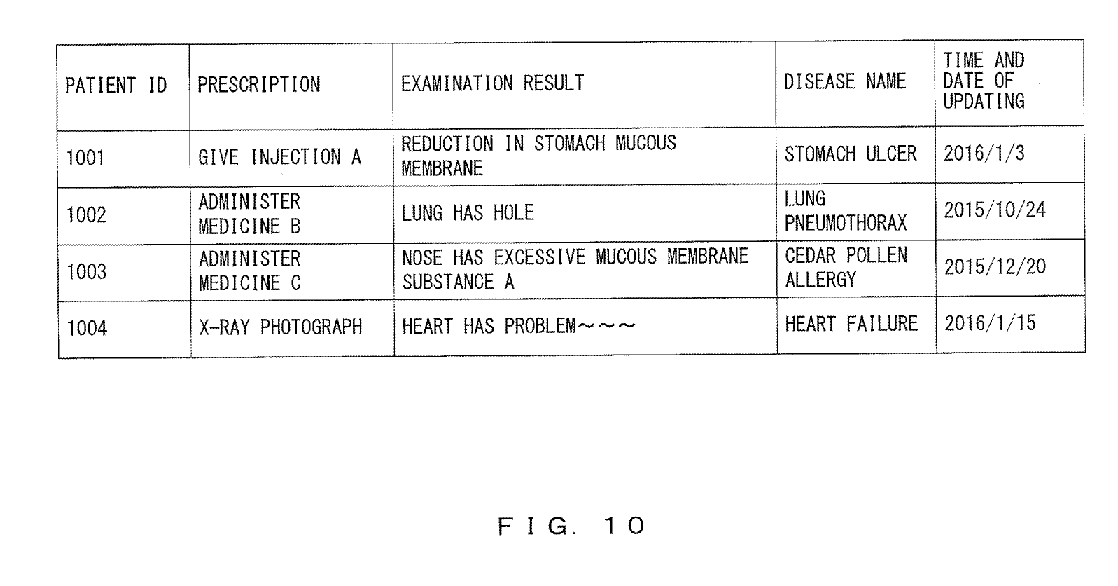

FIG. 10 illustrates an example of a consultation table included in the personal information 531 and the personal information 611. The consultation table illustrated in FIG. 10 is a table registering consultation information of patients and includes items of patient ID, prescription, examination result, disease name, and time and date of updating. Prescription represents a prescription given through a consultation, an examination result represents an examination result that was referred to for the consultation, and disease name represents the name of a disease determined in the consultation. A time and date of updating represents a time and date at which consultation information of each patient was updated.

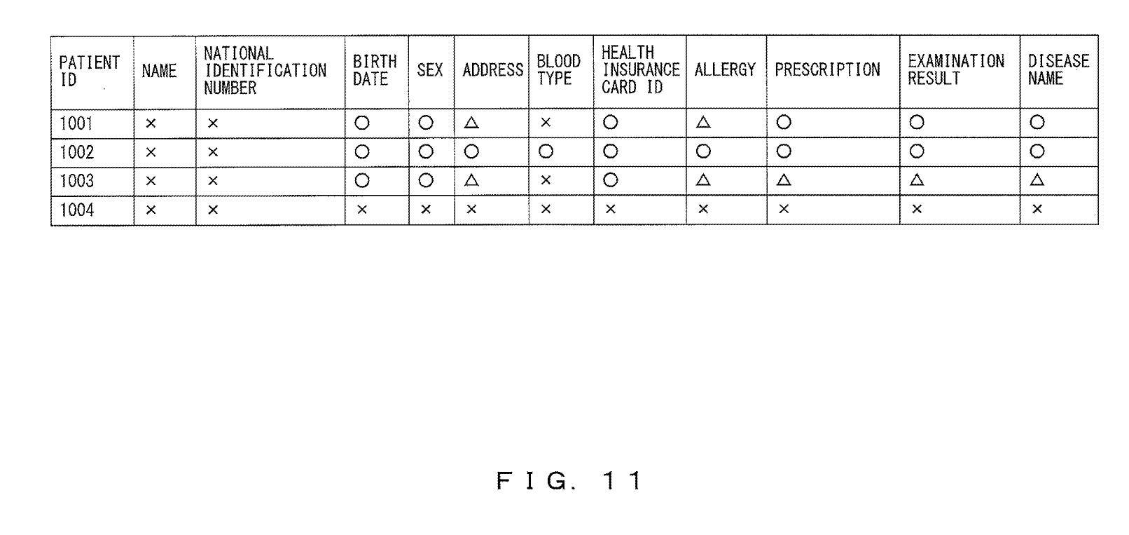

FIG. 11 illustrates an example of the confidentialization level information 612 used in mode M1. Each entry of the confidentialization level information 612 illustrated in FIG. 11 corresponds to personal information of each patient included in the basic table illustrated in FIG. 9 and the consultation table illustrated in FIG. 10, and includes one of the symbols of “∘”, “Δ” and “x” for each item. Among the symbols, “Δ” and “x” specify a confidentialization operation that is applied to each item included in personal information.

“∘” represents information that can be provided without being confidentialized, “Δ” represents information that can be provided when it is processed so that the individual person is not identified, and “x” represents information that is not provided at all.

To the information of an item for which “Δ” is set, a confidentialization operation is applied in which the information is converted into simplified information by using the process table 713. In such a case, when the process table 713 having different content is used, a confidentialization operation of a different confidentialization level is applied. To the information of an item for which “x” is set, a confidentialization operation of converting the information into data indicating that the information of the item has been confidentialized is applied.

In the information processing system 401, scopes of information that can be provided and methods of providing information may vary depending upon each patient's attitude toward personal information or the characteristics of the disease of each patient. For example, the confidentialization level information having a patient ID of “1001” has “∘” set for the birth date, the sex, the health insurance card ID, the prescription, the examination result and the disease name. Also, the information has “x” set for the name, the national identification number and the blood type, and has “Δ” set for the address and the allergy. By contrast, the confidentialization level information having a patient ID of “1004” has “x” set for all the items.

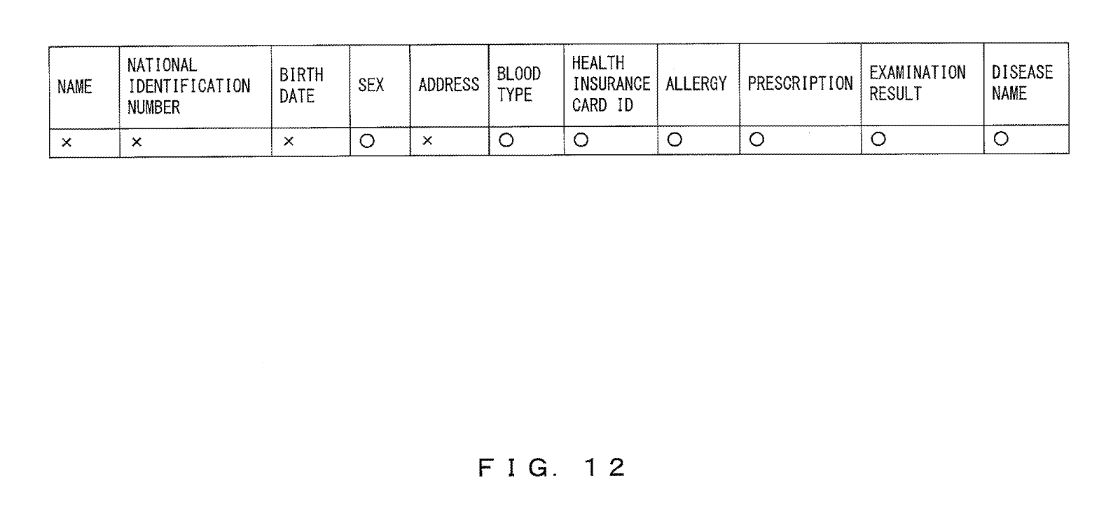

FIG. 12 illustrates an example of the confidentialization level information 715 used in mode M2. The confidentialization level information 715 illustrated in FIG. 12 is applied to the personal information 611 of all patients. In this example, “∘” is set for the sex, the blood type, the health insurance card ID, the allergy, the prescription, the examination result and the disease name, while “x” is set for the name, the national identification number, the birth date and the address.

It is also possible to set “Δ” as the confidentialization level information 715. “Δ” and “x” specify a confidentialization operation applied to each item included in personal information. To the information of an item for which “Δ” is set, a confidentialization operation is applied in which the information is converted into simplified information by using the process table 714. In such a case, when the process table 714 having different content is used, a confidentialization operation of a different confidentialization level is applied.

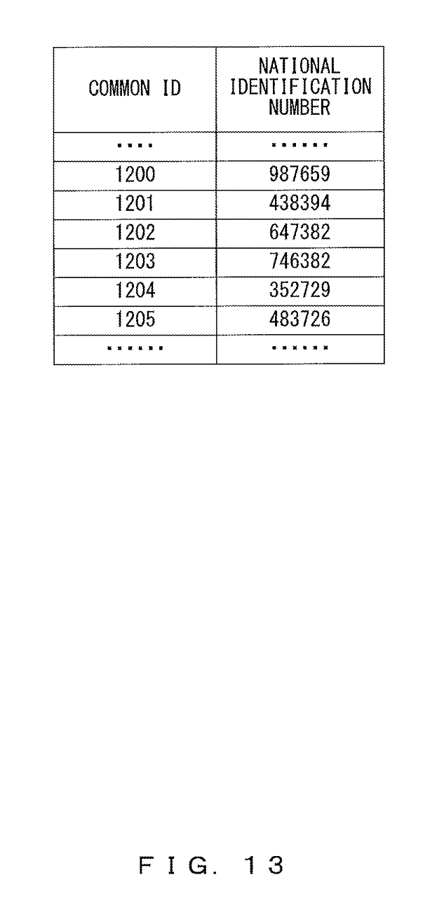

FIG. 13 illustrates an example of the ID table 426. The ID table 426 illustrated in FIG. 13 includes common IDs and national identification numbers and represents correspondence relationships for associating national identification numbers, which are personal IDs included in the personal information 611, and common IDs.

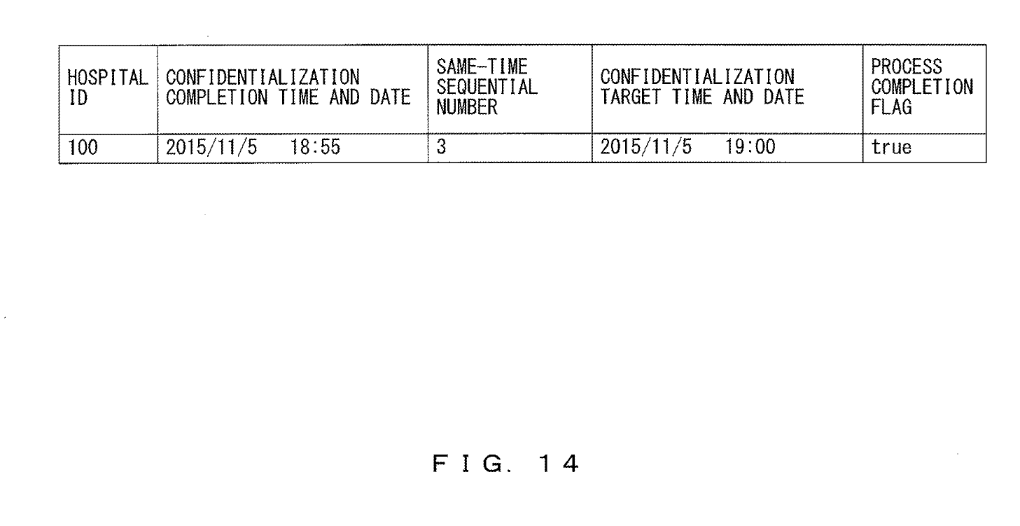

FIG. 14 illustrates an example of the time-date table 711 and the time-date table 712. The time-date table illustrated in FIG. 14 includes a hospital ID, a confidentialization completion time and date, a same-time sequential number, a confidentialization target time and date and a process completion flag. A hospital ID is an ID for identifying a hospital, and a confidentialization completion time and date is a time and date that represents the progress of a confidentialization process for the personal information 611. Each time the personal information 611 of one patient in the basic table of FIG. 9 and the consultation table of FIG. 10 is confidentialized for example, the time and date of updating of that piece of the personal information 611 is copied into the confidentialization completion time and date.

A same-time sequential number represents an order of the piece of the personal information 611 for which a confidentialization process has been completed from among a plurality of pieces of the personal information 611 that have the same time and date of updating. A same-time sequential number of “3” for example represents that a confidentialization process has been completed for up to the third piece of the personal information 611 from among the plurality of pieces of the personal information 611 having the time and date of updating copied into the confidentialization completion time and date. In such a case, a confidentialization process has not been completed for the fourth and subsequent pieces of the personal information 611.

A confidentialization target time and date is a time and date that specifies a scope of the personal information 611 that is a target of a confidentialization process. Pieces of the personal information 611 having a time and date of updating that is the same as or earlier than the confidentialization target time and date become a target of a confidentialization process. A process completion flag represents whether or not a confidentialization process has been completed for the pieces of the personal information 611 that are earlier than the confidentialization target time and date in each hospital. When a confidentialization target time and date is set in the time-date table, the process completion flag is set to “false”, and when a confidentialization process has been completed for the pieces of the personal information 611 that are earlier than the confidentialization target time and date, the process completion flag is set to “true”.

When a collection period of the personal information 611 is specified by a request from an information analysis institution in mode M2, the time-and-date management unit 703 sets the confidentialization completion time and date and the confidentialization target time and date of the time-date table 712 on the basis of the collection starting time and date and the collection ending time and date.

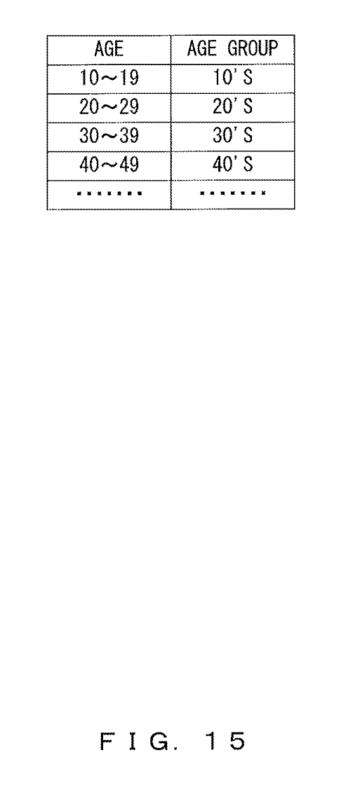

FIG. 15 illustrates an example of the process table 713 and the process table 714. The process table illustrated in FIG. 15 includes ages and age groups and represents correspondence relationships for associating ages, which are information before conversion, and age groups, which are information after conversion. An age can be calculated from the birth date included in the basic table illustrated in FIG. 9. By using the process table illustrated in FIG. 15, information of birth dates, which can be used for identifying persons, is simplified to information of age groups, which are anonymous data.

Also, when an item to be simplified is an address, a process table can also be used that is for deleting, from the character string of that address, information of the name of the city, the block number, etc., which can be used for identifying the person, so as to simplify the character string. This makes it possible to simplify the address of “1-24-2, Kounan-cho, Kita-ku, Yokohama city” of FIG. 9 to “Yokohama city”.

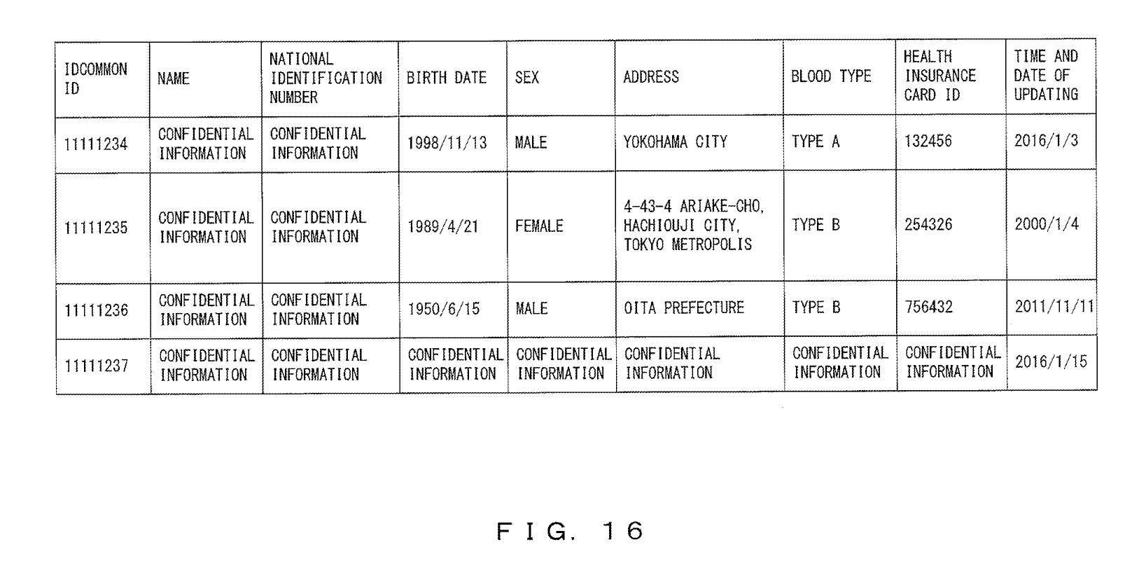

FIG. 16 illustrates an example of a basic table included in the confidentialized personal information 811 and the confidentialized personal information 812. The basic table illustrated in FIG. 16 includes items of common ID, name, national identification number, birth date, sex, address, blood type, health insurance card ID and time and date of updating. A common ID is a common ID assigned by the identification information assignment unit 425.

In this example, the names and the national identification numbers of all the patients have been converted into the character string “confidential information”, which is data indicating that the information has been confidentialized. Also, the address of the patient having the common ID “11111234” has been converted into “Yokohama city” as a simplified character string, and the information of all the items of the patient having the common ID “11111237” has been converted into the character string “confidential information”.

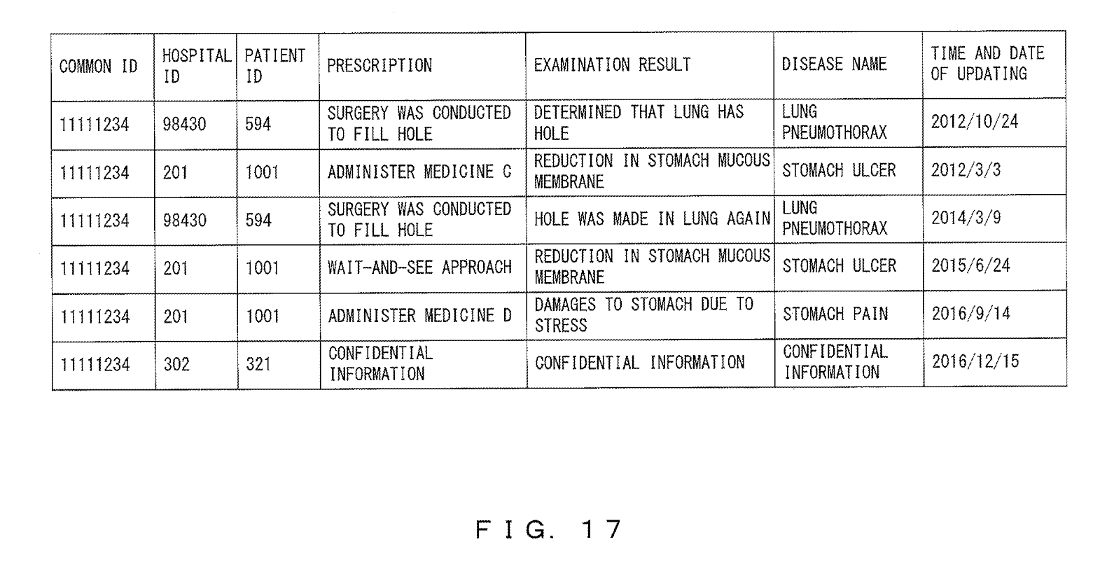

FIG. 17 illustrates an example of a consultation table included in the confidentialized personal information 811 and the confidentialized personal information 812. The consultation table illustrated in FIG. 17 includes items of common ID, hospital ID, patient ID, prescription, examination result, disease name and time and date of updating.

In this example, the patient having the common ID “11111234” has been registered as the patient having the patient ID “594” in the hospital having the hospital ID “98430” and has been registered as the patient having the patient ID “1001” in the hospital having the hospital ID “201”. Also, the same patient has been registered as the patient having the patient ID “321” in the hospital having the hospital ID “302”. Also, the prescription, the examination result and the disease name have been converted into the character string “confidential information” in the hospital having the hospital ID “302”.

As described above, assigning a common ID to the confidentialized personal information 811 and the confidentialized personal information 812 makes it possible to determine pieces of information of the same patient from among pieces of confidentialized personal information collected from a plurality of hospitals.

Incidentally, the respective hospitals do not always have the personal information 611 in the same data format. When hospitals have the personal information 611 in different data formats, the confidentialization unit 702 converts the data formats of the pieces of the personal information 611 into a uniform data format and generates the confidentialized personal information 811 and the confidentialized personal information 812 from the converted personal information. This makes it possible to compensate for differences in data formats between hospitals.

For example, the server 503 of each hospital system 411-i generates a conversion program for converting the data format of the personal information 531 in the operation DB 511 into a uniform data format and transmits the program to the backup system 412 in advance. Then, the confidentialization unit 702 of the VM 424-i uses the received conversion program to convert the data format of the personal information 611 into the uniform data format.

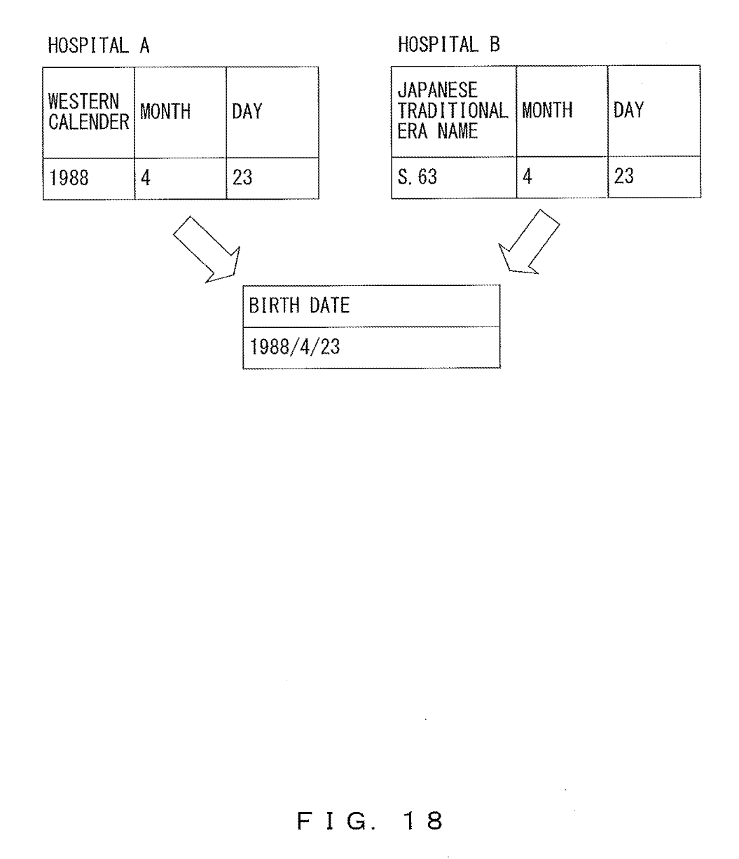

FIG. 18 illustrates an example of a process of converting a data format. In this example, a “year”, “month” and “day” on which the patient was born are respectively described in separate columns as information of the birth date in the personal information 611 of hospitals A and B. Among them, the personal information 611 of hospital A has the information of “year” described in the Western calendar, while the personal information 611 of hospital B has the same information described in the Japanese traditional era name.

When the personal information 611 of hospital A is to be converted, the confidentialization unit 702 reads character strings from the respective columns of “year (Western calendar)”, “month” and “day” in the personal information 611. Then, the confidentialization unit 702 uses the conversion program received from the server 503 of hospital A to connect the character strings to each other with slashes or “/” and generates the character string “birth date” in the uniform data format.

When the personal information 611 of hospital B is to be converted, the confidentialization unit 702 reads character strings from the respective columns of “year (Japanese traditional era name), “month” and “day” in the personal information 611. Then, the confidentialization unit 702 uses the conversion program received from the server 503 of hospital B to convert the character string of the year in the Japanese traditional era name into a character string in the Western calendar and connects the character strings to each other with slashes or “/”, and thereby generates the character string “birth date” in the uniform data format.

When the information processing system 401 illustrated in FIG. 4 operates in mode M1, an electronic medical record is analyzed in for example procedures similar to (P11) through (P23) described above. When the information processing system 401 operates in mode M2, an electronic medical record is analyzed in for example the following procedures.

(P51) An analyst of an information analysis institution uses the PC 432 to transmit, to the VM 424-i, an information provision request together with the process table 714 and the confidentialization level information 715 specified by the information analysis institution.

(P52) The confidentialization unit 702 of the VM 424-i switches the process table that it refers to in a confidentialization process from the process table 713 to the process table 714.

(P53) The confidentialization unit 702 switches the confidentialization level information that it refers to in a confidentialization process from the confidentialization level information 612 to the confidentialization level information 715.

(P54) The VM 424-i requests that hospital system 411-i of each hospital transfer, to the backup storage device 421-i, the personal information 611 that is the latest as of the moment at which an information provision request was received from the information analysis institution.

(P55) The hospital system 411-i transfers the personal information 611 to the backup storage device 421-i in response to the request from the VM 424-i.

(P56) The search unit 804 of the collection storage device 433 compares the collection period specified by the information provision request and the time and date of updating in each entry in the confidentialized personal information 811 that is already stored in the collection DB 801.

(P57) The search unit 804 generates a copy of an entry that is within a period overlapping the collection period specified by the information provision request and stores the copy in the collection DB 802 as the confidentialized personal information 812. Thereby, the confidentialized personal information 811 generated in mode M1 can be reused in an analysis process in mode M2.

(P58) The comparison unit 701 of the VM 424-i compares the confidentialization level information 612 in the backup DB 602 and the confidentialization level information 715 received from the information analysis institution and generates a comparison result.

(P59) In accordance with the comparison result generated by the comparison unit 701, the confidentialization unit 702 applies a confidentialization process to an entry of the personal information 611 that is within the overlapping period, and thereby generates the confidentialized personal information 812.

(P60) The transfer unit 704 transfers the confidentialized personal information 812 to the collection storage device 433.

(P61) The collection storage device 433 overwrites the confidentialized personal information 812 in the collection DB 802 with the received confidentialized personal information 812.

(P62) The confidentialization unit 702 applies a confidentialization process to an entry that is within a period that has not received the collection conducted by the collection storage device 433 in the personal information 611 that is within the collection period, and thereby generates the confidentialized personal information 812.

(P63) The transfer unit 704 transfers the confidentialized personal information 812 to the collection storage device 433.

(P64) The collection storage device 433 stores the received confidentialized personal information 812 in the collection DB 802.

(P65) The analyst of the information analysis institution uses the PC 432 to analyze the confidentialized personal information 812 and stores the analysis result in the server 431.

In (P59) through (P61), the confidentialization unit 702 applies a confidentialization process to the personal information 611 and the collection storage device 433 modifies the confidentialized personal information 812 in the collection DB 802 in accordance with for example the following criteria.

(C1) Item having “∘” as the confidentialization level information 715 of the information analysis institution and having “∘” as the confidentialization level information 612 of the backup DB 602

The confidentialization unit 702 does not apply a confidentialization operation to the information of such an item, the transfer unit 704 does not transfer confidentialized information of such an item, and the collection storage device 433 does not modify confidentialized information of such an item in the collection DB 802.

(C2) Item having “∘” as the confidentialization level information 715 of the information analysis institution and having “Δ” or “x” as the confidentialization level information 612 of the backup DB 602

The confidentialization unit 702 does not apply a confidentialization operation to the information of such an item, the transfer unit 704 transfers the information of such an item as it is, and the collection storage device 433 overwrites the confidentialized information of the item in the collection DB 802 with received information.

(C3) Item having “Δ” as the confidentialization level information 715 of the information analysis institution and having “∘” as the confidentialization level information 612 of the backup DB 602

The confidentialization unit 702 applies a confidentialization operation of “Δ” to the information of such an item by using the process table 714, and the transfer unit 704 transfers the confidentialized information of the item. The collection storage device 433 overwrites the confidentialized information of the item in the collection DB 802 with the received confidentialized information.

(C4) Item having “Δ” as the confidentialization level information 715 of the information analysis institution and having “Δ” as the confidentialization level information 612 of the backup DB 602 and having a process table 714 that is the same as the process table 713

The confidentialization unit 702 does not apply a confidentialization operation to the information of such an item, the transfer unit 704 does not transfer confidentialized information of such an item, and the collection storage device 433 does not modify confidentialized information of such an item in the collection DB 802.

(C5) Item having “Δ” as the confidentialization level information 715 of the information analysis institution and having “Δ” as the confidentialization level information 612 of the backup DB 602 and having a process table 714 that is different from the process table 713

The confidentialization unit 702 applies a confidentialization operation of “Δ” to the information of such an item by using the process table 714, and the transfer unit 704 transfers the confidentialized information of the item. The collection storage device 433 overwrites the confidentialized information of the item in the collection DB 802 with the received confidentialized information.

(C6) Item having “Δ” as the confidentialization level information 715 of the information analysis institution and having “x” as the confidentialization level information 612 of the backup DB 602

The confidentialization unit 702 applies a confidentialization operation of “Δ” to the information of such an item by using the process table 714, and the transfer unit 704 transfers the confidentialized information of the item. The collection storage device 433 overwrites the confidentialized information of the item in the collection DB 802 with the received confidentialized information.

(C7) Item having “x” as the confidentialization level information 715 of the information analysis institution and having “∘” or “Δ” as the confidentialization level information 612 of the backup DB 602

The confidentialization unit 702 applies a confidentialization operation of “x” to the information of such an item, the transfer unit 704 transfers the confidentialized information of the item, and the collection storage device 433 overwrites the confidentialized information of the item in the collection DB 802 with the received confidentialized information.

(C8) Item having “x” as the confidentialization level information 715 of the information analysis institution and having “x” as the confidentialization level information 612 of the backup DB 602

The confidentialization unit 702 does not apply a confidentialization operation to the information of such an item, the transfer unit 704 does not transfer confidentialized information of such an item, and the collection storage device 433 does not modify confidentialized information of such an item in the collection DB 802.

According to the information processing system 401 as described above, it is possible to reuse the confidentialized personal information 811 that has already been stored in the collection DB 801, for a period that is a target of a confidentialization process in mode M1 and that is included in a collection period specified by an information analysis institution. Accordingly, only the confidentialized personal information 812 that is not a target of a confidentialization process in mode M1 and a modified portion of the reused confidentialized personal information 811 are transferred from the backup system 412 to the analysis system 413.

This can reduce the amount of data of the confidentialized personal information 812 transferred from the backup system 412 to the analysis system 413 in a confidentialization process in mode M2. Accordingly, the loads on a communication network between the backup system 412 and the analysis system 413 are reduced, increasing the performance and stability in comparison with the information processing system 101 illustrated in FIG. 1.

Also, a confidentialization process is again performed in the VM 424-i for an item that is included in the confidentialized personal information 811 having already been stored in the collection DB 801 and that received a confidentialization operation on a level that is different from the confidentialization level requested by an information analysis institution. This makes it possible to store, in the collection DB 802, the confidentialized personal information 812 corresponding to a confidentialization level requested by an information analysis institution even when the confidentialization level requested by the information analysis institution is different from a confidentialization level specified by a patient.

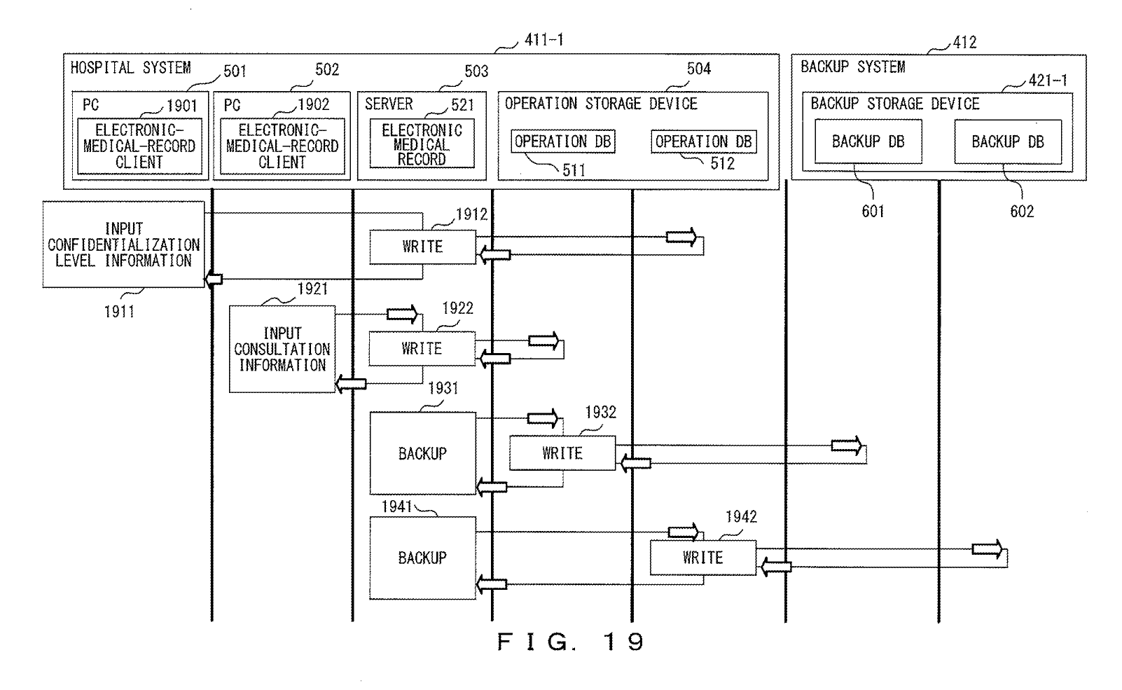

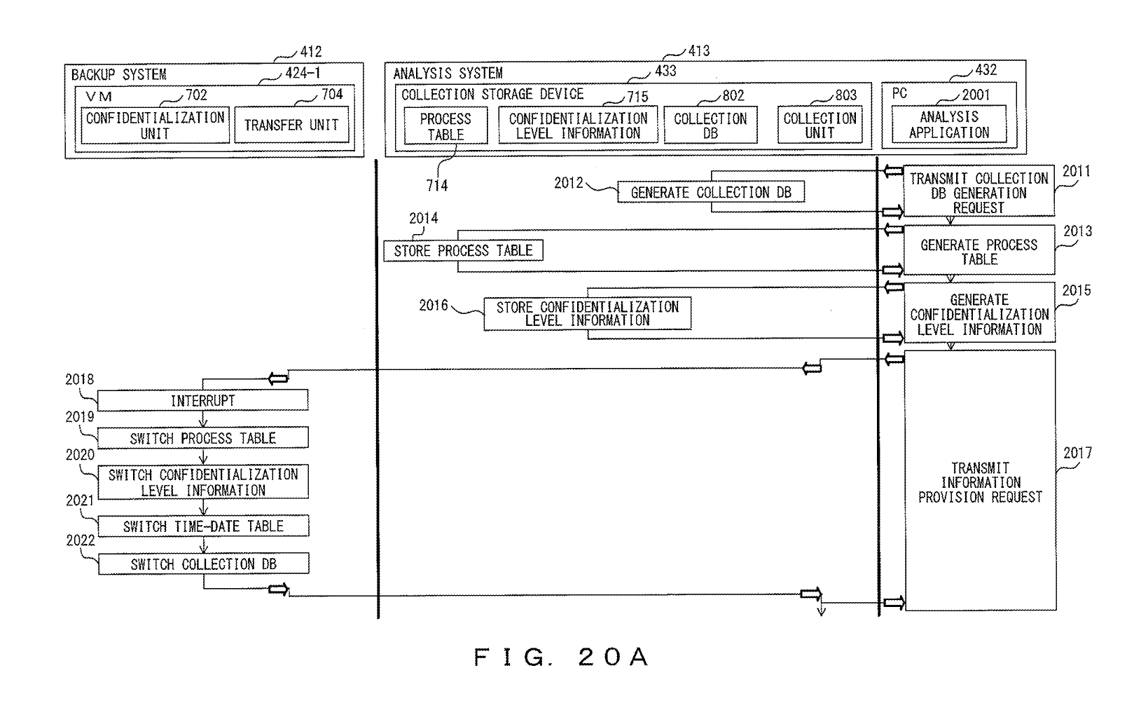

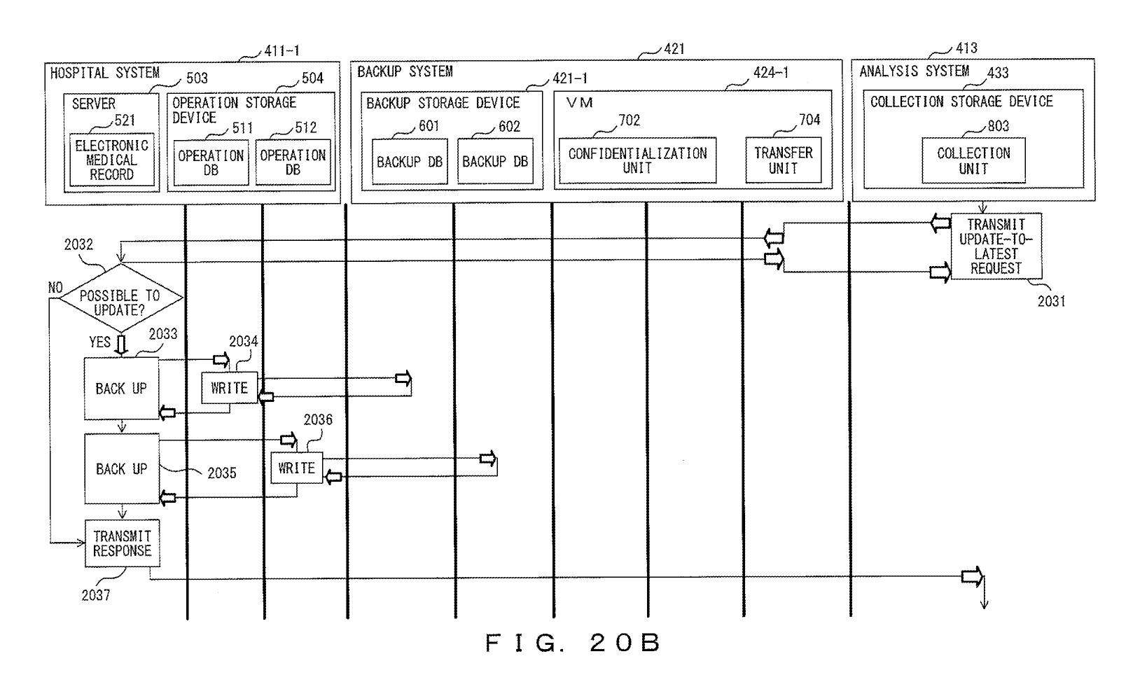

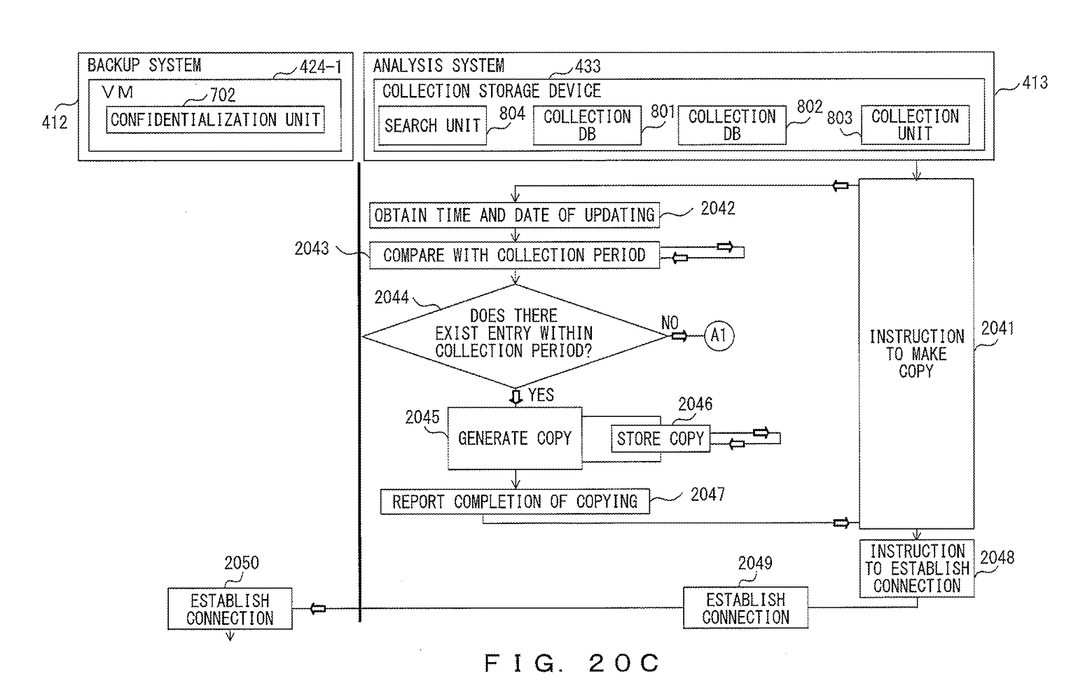

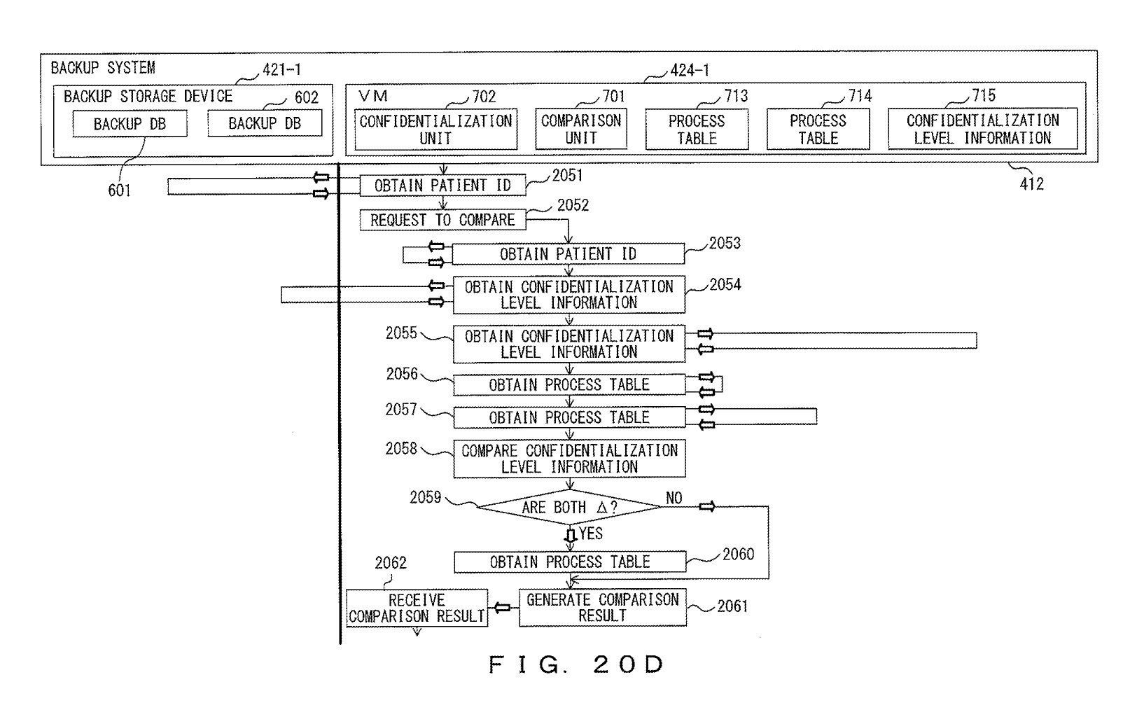

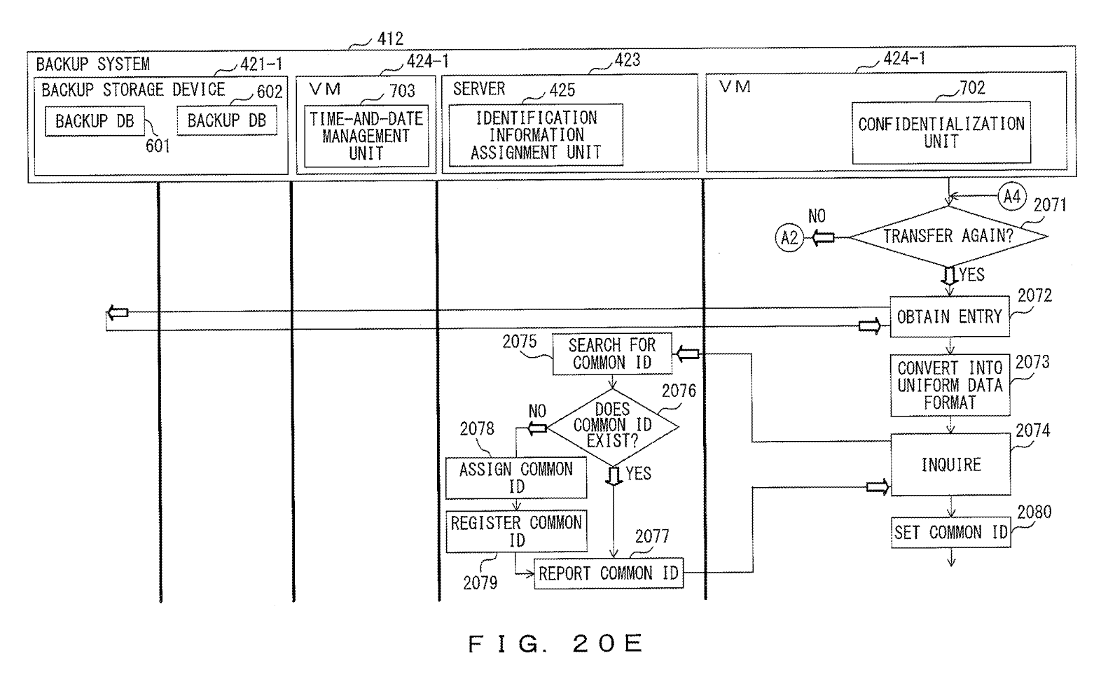

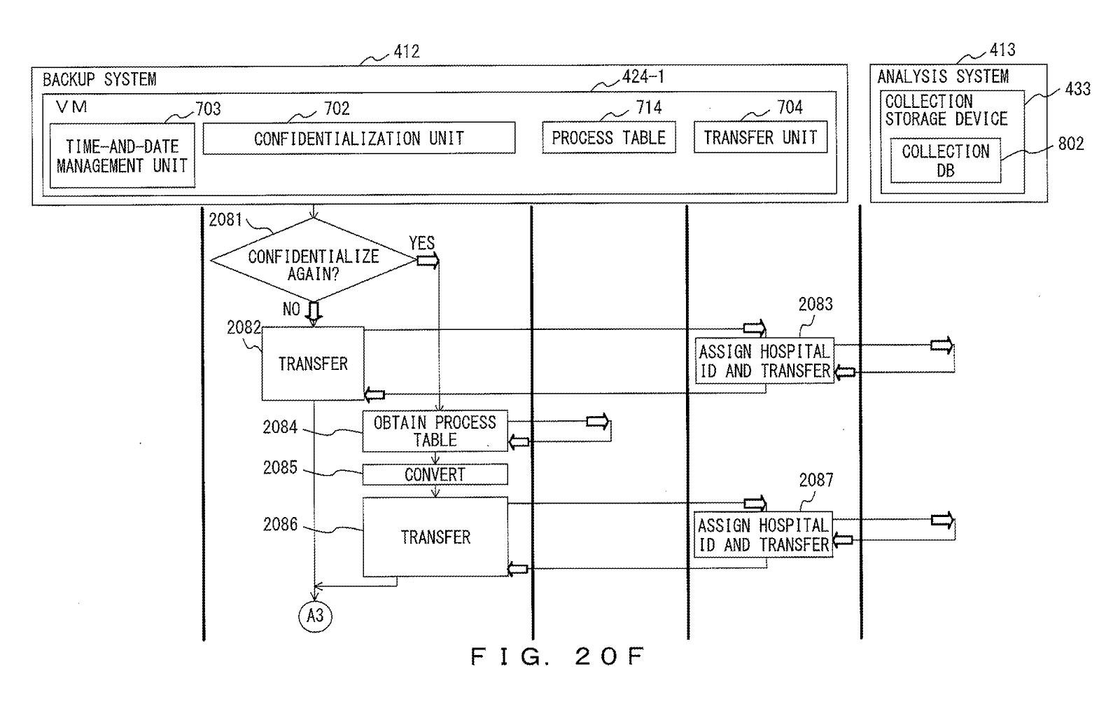

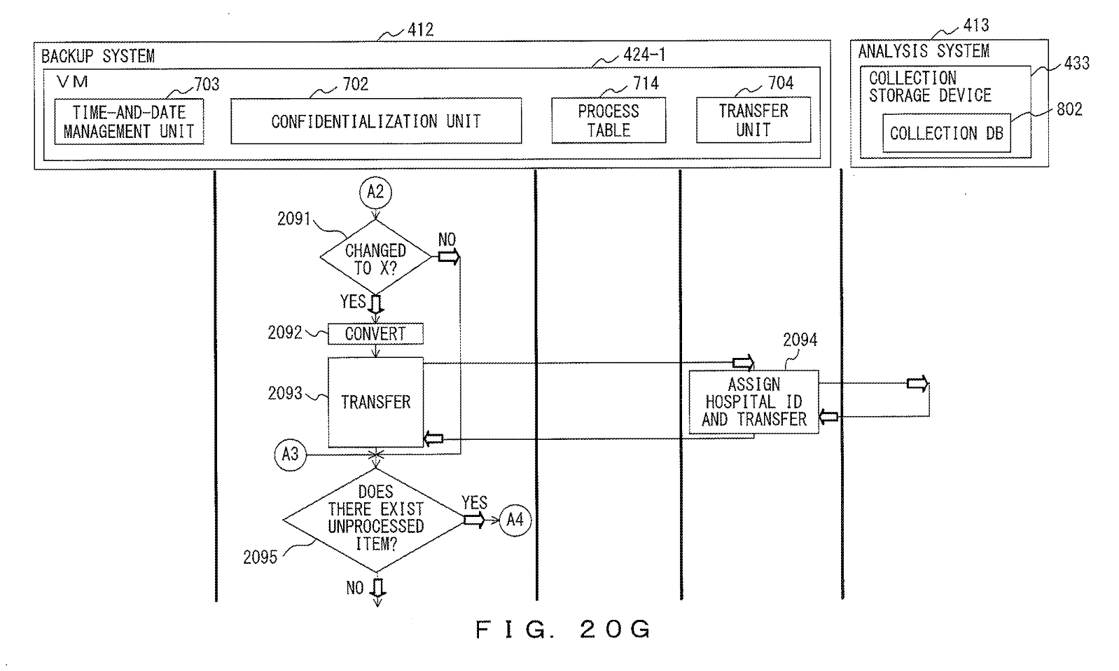

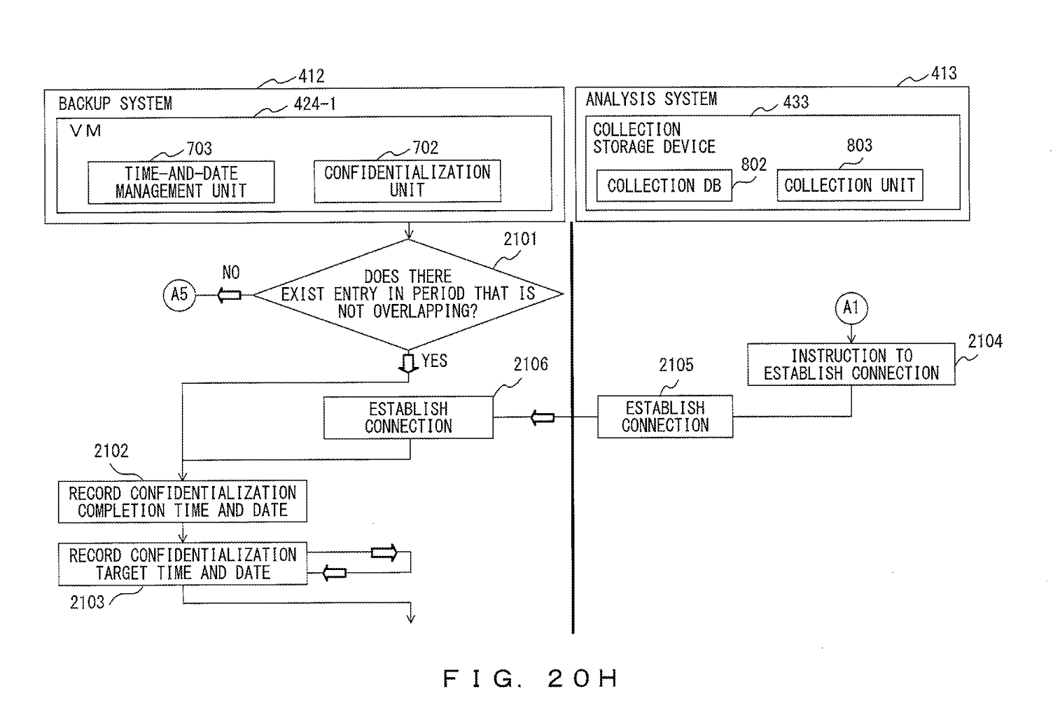

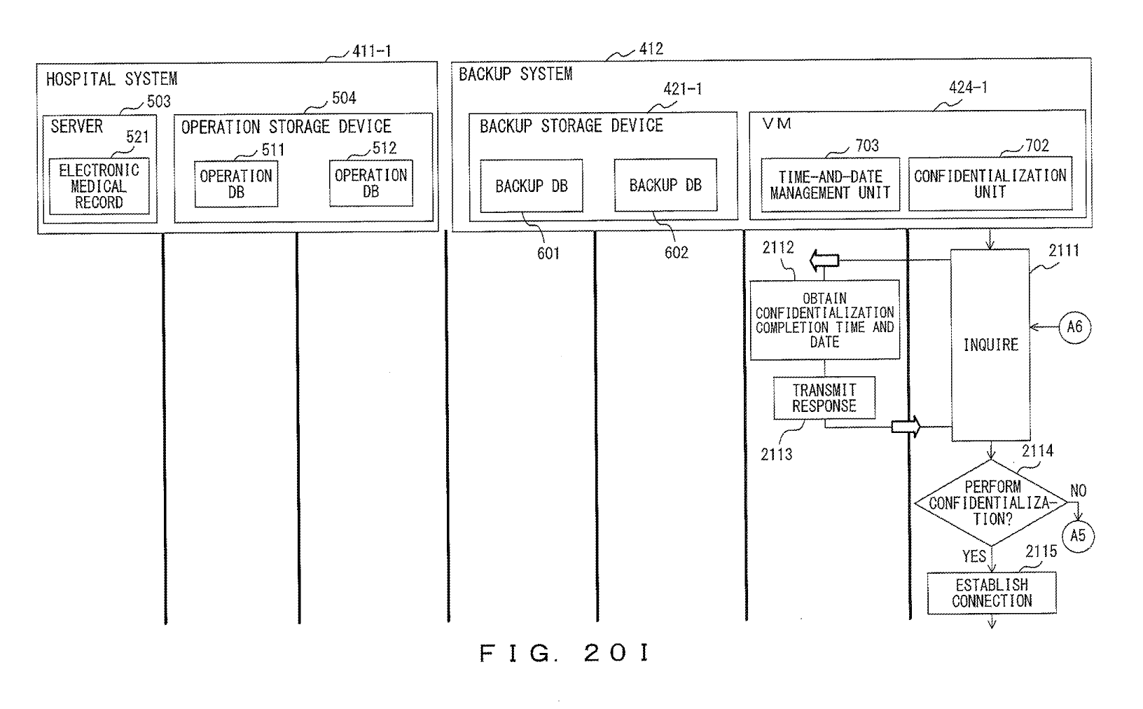

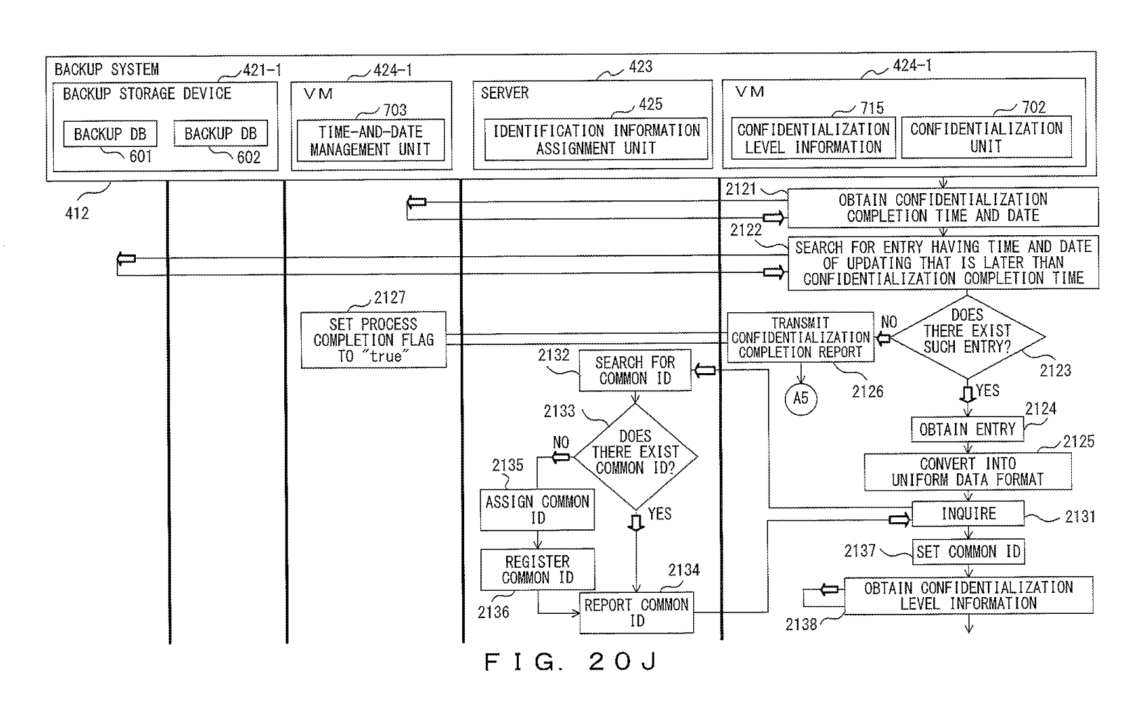

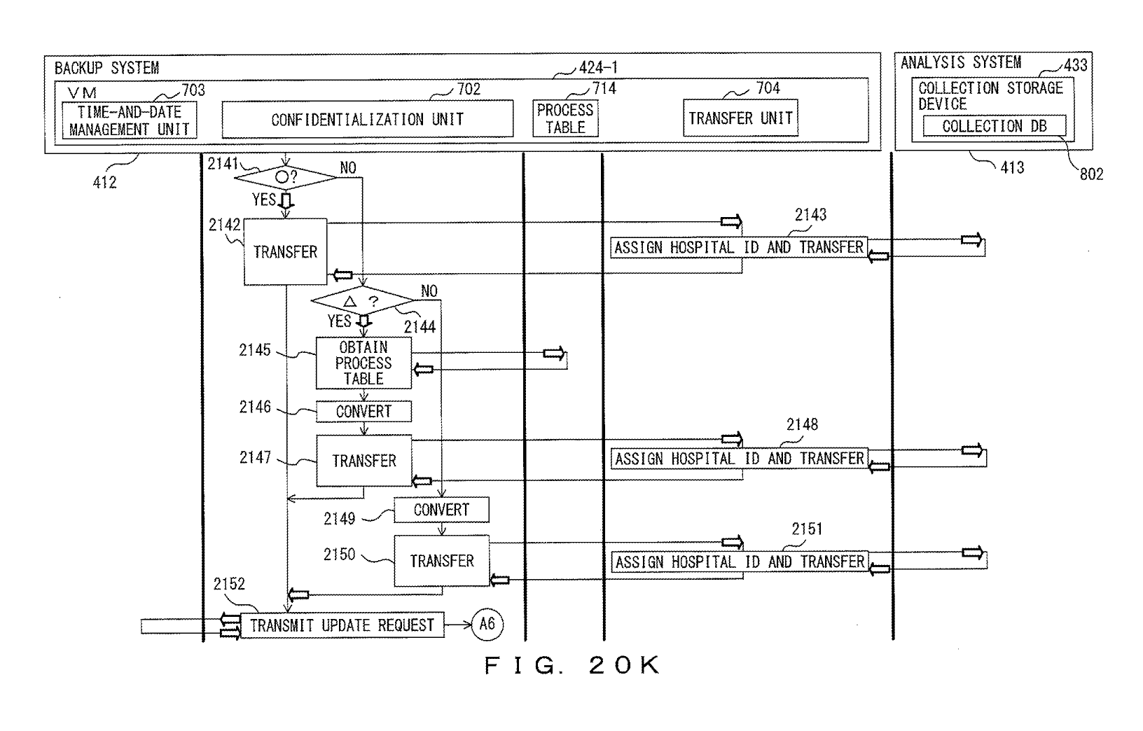

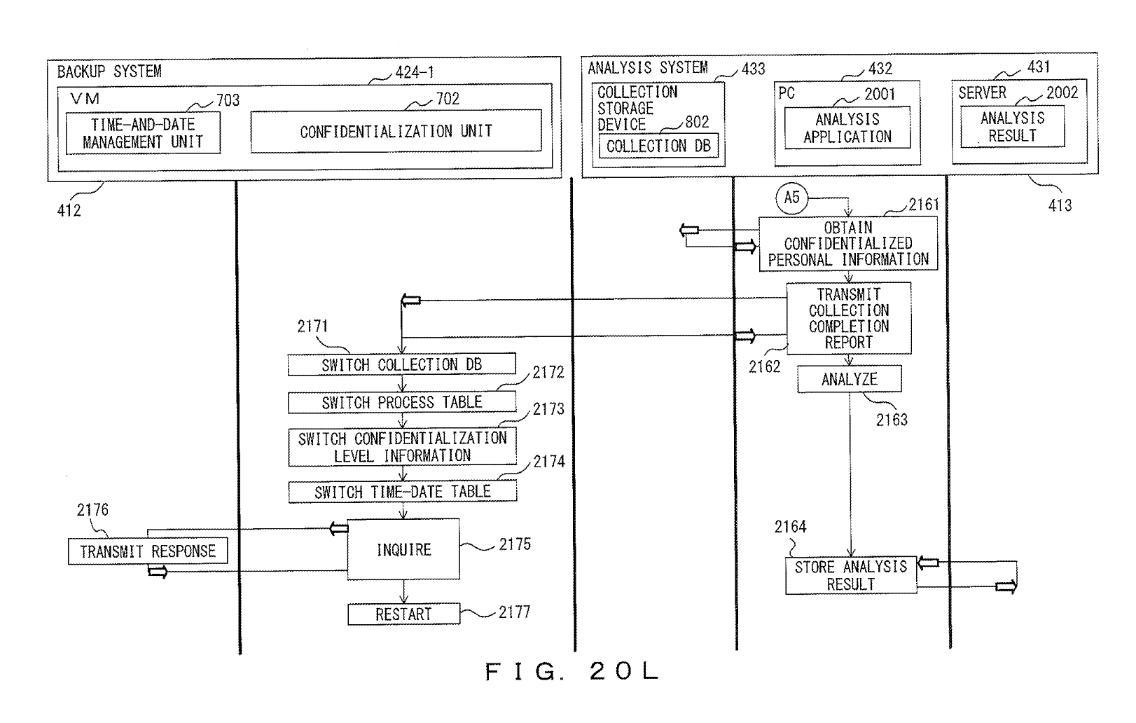

Next, more detailed explanations will be given for operations of the information processing system 401 illustrated in FIG. 4 by referring to FIG. 19 and FIG. 20A through FIG. 20L.

FIG. 19 illustrates an information provision sequence in mode M1. The PCs 501 and 502 of the hospital system 411-1 have electronic-medical-record clients 1901 and 1902 installed as applications in them, respectively.

First, in accordance with a manipulation conducted by a clerk or a patient, the electronic-medical-record client 1901 inputs confidentialization level information specified by the patient to the electronic medical record 521 of the server 503 (step 1911). Then, the server 503 writes the confidentialization level information that has been input to the electronic medical record 521 to the operation DB 512 of the operation storage device 504 as the confidentialization level information 532 (step 1912).

Next, the electronic-medical-record client 1902 inputs the consultation information of the patient to the electronic medical record 521 on the basis of the manipulation conducted by a doctor (step 1921). Next, the server 503 writes the consultation information that was input to the electronic medical record 521 to the operation DB 511 of the operation storage device 504 as the personal information 531 (step 1922).

Thereafter, a system administrator of each hospital periodically makes a backup. Then, the server 503 transmits an instruction to make a backup of the personal information 531 to the operation storage device 504 (step 1931). Thereafter, the operation storage device 504 writes a copy of the personal information 531 to the backup DB 601 of the backup storage device 421-1 as the personal information 611 (step 1932).

Next, the server 503 transmits an instruction to make a backup of the confidentialization level information 532 to the operation storage device 504 (step 1941). Then, the operation storage device 504 writes a copy of the confidentialization level information 532 to the backup DB 602 of the backup storage device 421-1 as the confidentialization level information 612 (step 1942).

Also in the hospital systems 411-2 through 411-M, the personal information 531 and the confidentialization level information 532 are written to the operation storage device 504 through an information provision sequence similar to that illustrated in FIG. 19. Then, the personal information 611 and the confidentialization level information 612 are written to the backup storage devices 421-2 through 421-M.

For example, the information processing system 401 performs a confidentialization process in mode M1 in a normal state and preferentially performs a confidentialization process in mode M2 when receiving a request from an information analysis institution at a time of emergency. In such a case, the information processing system 401 interrupts the confidentialization processes in mode M1 for all the hospitals and starts confidentialization processes in mode M2.

FIG. 20A through FIG. 20L illustrate an example of an operation sequence in mode Mode M2. First, an analysis application 2001 of the analysis system 413 transmits a collection DB generation request to the collection storage device 433 on the basis of a manipulation conducted by an analyst of an information analysis institution (step 2011). The collection storage device 433 generates the collection DB 802 (step 2012).

Next, the analysis application 2001 generates the process table 714 and transmits the table to the collection storage device 433 on the basis of the manipulation conducted by the analyst (step 2013). The collection storage device 433 stores the received process table 714 (step 2014).

Next, the analysis application 2001 generates the confidentialization level information 715 and transmits the information to the collection storage device 433 on the basis of the manipulation conducted by the analyst (step 2015). The collection storage device 433 stores the received confidentialization level information 715 (step 2016).

Next, on the basis of the manipulation conducted by the analyst, the analysis application 2001 transmits, to the VM 424-1 of the backup system 412, an information provision request including a collection period together with the process table 714 and the confidentialization level information 715.

The confidentialization unit 702 of the VM 424-1 interrupts the confidentialization process in mode M1 (step 2018) and switches the process table that it refers to in a confidentialization process from the process table 713 to the process table 714 (step 2019). Next, the confidentialization unit 702 switches the confidentialization level information that it refers to in a confidentialization process from the confidentialization level information 612 to the confidentialization level information 715 (step 2020).

Next, the confidentialization unit 702 switches the time-date table that it refers to in a confidentialization process from the time-date table 711 to the time-date table 712 (step 2021). Upon doing this, the time-and-date management unit 703 sets a time and date that is earlier than the collection starting time and date of the collection period included in the information provision request as a confidentialization completion time and date of the time-date table 712 and sets the collection ending time and date as the confidentialization target time and date. Then, the time-and-date management unit 703 sets the process completion flag to “false”.

Next, the confidentialization unit 702 switches the transfer destination of confidentialized personal information from the collection DB 801 to the collection DB 802 (step 2022).

Next, the collection unit 803 of the collection storage device 433 transmits an update-to-latest request of the backup DB 601 to the backup system 412 (step 2031), and the transfer unit 704 transfers the update-to-latest request to the hospital system 411-1.

The server 503 of the hospital system 411-1 determines whether or not it is possible to update the backup DB 601 to the latest state (step 2032). The server 503 determines that it is possible to perform updating to the latest state when the personal information 611 of the backup DB 601 is not the latest and the personal information 611 can be backed up immediately. Also, the server 503 determines that it is not possible to perform updating to the latest state when the personal information 611 of the backup DB 601 is the latest or when it is not possible to back up the personal information 611 immediately.

When updating to the latest state is possible (YES in step 2032), the server 503 transmits an instruction to make a backup of the personal information 531 to the operation storage device 504 (step 2033). Then, the operation storage device 504 writes a copy of the personal information 531 to the backup DB 601 of the backup storage device 421-1 as the personal information 611 (step 2034).

Next, the server 503 transmits an instruction to make a backup of the confidentialization level information 532 to the operation storage device 504 (step 2035). Then, the operation storage device 504 writes a copy of the confidentialization level information 532 to the backup DB 602 of the backup storage device 421-1 as the confidentialization level information 612 (step 2036).

Then, the server 503 transmits, to the collection unit 803, a response indicating the completion of updating to the latest state (step 2037). When updating to the latest state is not possible (NO in step 2032), the server 503 immediately transmits a response indicating the completion of updating to the latest state to the collection unit 803 (step 2037).

Next, the collection unit 803 instructs the search unit 804 to make a copy of the confidentialized personal information 811 in the collection DB 801 (step 2041). The search unit 804 obtains a time and date of updating from the confidentialized personal information 811 in the collection DB 801 (step 2042). Then, the search unit 804 compares the obtained time and date of updating with the collection period included in the information provision request (step 2043) and checks whether or not there exists an entry of the confidentialized personal information 811 having a time and date of updating that is within the collection period (step 2044).

When there exists an entry having a time and date of updating that is within the collection period (YES in step 2044), the search unit 804 generates a copy of that entry (step 2045) and stores the copy in the collection DB 802 as the confidentialized personal information 812 (step 2046). Then, the search unit 804 reports the completion of the copying to the collection unit 803 (step 2047).

Next, the collection unit 803 instructs the VM 424-1 to establish a connection between the collection DB 802 and the confidentialization unit 702 (step 2048). Then, the collection unit 803 establishes a connection between the collection DB 802 and the confidentialization unit 702 (step 2049), and the VM 424-1 also establishes a connection between the collection DB 802 and the confidentialization unit 702 (step 2050).

Next, the confidentialization unit 702 obtains a patient ID from an entry that has a time and date of updating within the collection period and that is included in the personal information 611 in the backup DB 601 (step 2051). Then, the confidentialization unit 702 requests that the comparison unit 701 compare the confidentialization level information 612 in the backup DB 602 and the confidentialization level information 715 received from the analysis system 413 (step 2052).

The comparison unit 701 obtains the patient ID from the confidentialization unit 702 (step 2053) and obtains the confidentialization level information 612 corresponding to the obtained patient ID from the backup DB 602 (step 2054). Then, the comparison unit 701 obtains the confidentialization level information 715 from the memory 705 (step 2055), obtains the process table 713 from the memory 705 (step 2056) and obtains the process table 714 from the memory 705 (step 2057).

Next, for each patient ID that has been obtained, the comparison unit 701 compares the confidentialization level information 612 and the confidentialization level information 715 (step 2058) and checks whether or not the confidentialization level is “Δ” in both the confidentialization level information 612 and the confidentialization level information 715 (step 2059).

When the confidentialization level is “Δ” in both of the pieces of information (YES in step 2059), the comparison unit 701 compares the process table 713 and the process table 714 (step 2060). Then, the comparison unit 701 generates a comparison result so as to transfer the result to the confidentialization unit 702 (step 2061), and the confidentialization unit 702 receives the comparison result (step 2062). When the confidentialization level is not “Δ” in both or either of the pieces of information (NO in step 2059), the comparison unit 701 performs the process in step 2061.

The generated comparison result includes, for each patient ID and each item of the personal information 611, a combination of a confidentialization level specified by the confidentialization level information 612 and a confidentialization level specified by the confidentialization level information 715, and information indicating whether or not the process table 713 and the process table 714 are the same as each other.

In step 2051, patient IDs are obtained only from entries having a time and date of updating that is within a collection period, and thereby the confidentialization level information 612 that is a comparison target can be narrowed only to the confidentialization level information 612 of patients of such patient IDs. This reduces the amount of data that is a comparison target, improving the comparison process.

Next, for each item of each entry of the personal information 611 having a time and date of updating that is within a collection period, the confidentialization unit 702 refers to the comparison result and determines whether or not to again transfer the information of that item or information obtained by processing the information of that item to the collection storage device 433 (step 2071).

When the comparison result for an item that is a process target meets the condition of (C2), (C3), (C5) or (C6) described above, the confidentialization unit 702 determines that it will transfer the information again. When the comparison result for an item that is a process target meets the condition of (C1), (C4), (C7) or (C8) described above, the confidentialization unit 702 determines that it will not transfer the information again.

When the information is to be transmitted again (YES in step 2071), the confidentialization unit 702 obtains an entry that is a process target from the personal information 611 in the backup DB 601 (step 2072). Then, the confidentialization unit 702 uses the conversion program of the hospital system 411-1 to convert the data format of a process-target item of the obtained entry into the uniform data format (step 2073).

Next, the confidentialization unit 702 uses the personal ID included in the obtained entry to inquire of the identification information assignment unit 425 of the server 423 about a common ID corresponding to the personal ID (step 2074).

The identification information assignment unit 425 searches for a common ID corresponding to the personal ID (step 2075) and checks whether or not there exists such a common ID (step 2076). When a common ID corresponding to the personal ID exists (YES in step 2076), the identification information assignment unit 425 reports that common ID to the confidentialization unit 702 (step 2077).

When a common ID corresponding to the personal ID does not exist (NO in step 2076), the identification information assignment unit 425 assigns a new common ID to that personal ID (step 2078). Then, the identification information assignment unit 425 registers the correspondence relationship between that personal ID and the assigned common ID in the ID table 426 (step 2079) and reports the assigned common ID to the confidentialization unit 702 (step 2077).

Next, the confidentialization unit 702 sets the common ID reported from the identification information assignment unit 425 in the obtained entry (step 2080). Then, the confidentialization unit 702 refers to the comparison result and determines whether or not to again confidentialize the information of an item that is a process target (step 2081).

When the comparison result for an item that is a process target meets the condition of (C3), (C5) or (C6) described above, the confidentialization unit 702 determines that it will confidentialize the information again. When the comparison result for an item that is a process target meets the condition of (C2) described above, the confidentialization unit 702 determines that it will not confidentialize the information again.

When the information is not to be confidentialized again (NO in step 2081), the confidentialization unit 702 transfers the information of the item that is a process target to the transfer unit 704 as it is (step 2082). Then, the transfer unit 704 assigns the hospital ID to the received information and transfers the information to the collection storage device 433 of the analysis system 413 (step 2083). The collection storage device 433 overwrites the confidentialized information of the item that is a process target included in the confidentialized personal information 812 in the collection DB 802, with the information received from the transfer unit 704.

When the information is to be confidentialized again (YES in step 2081), the confidentialization unit 702 obtains the process table 714 (step 2084) and converts the information of the item that is a process target into simplified information by using the process table 714 (step 2085). Then, the confidentialization unit 702 transfers the information after the conversion to the transfer unit 704 (step 2086), and the transfer unit 704 assigns the hospital ID to the received information and transfers the information to the collection storage device 433 (step 2087). The collection storage device 433 overwrites the confidentialized information of the item that is a process target included in the confidentialized personal information 812 in the collection DB 802, with the information received from the transfer unit 704.

Next, the confidentialization unit 702 checks whether or not there exists an item that has not been processed in an entry having a time and date of updating that is within a collection period (step 2095). When there exists an item or entry that has not been processed (YES in step 2095), the confidentialization unit 702 repeats the processes in and after step 2071 for the next item.

When the information is not to be transferred again (NO in step 2071), the confidentialization unit 702 refers to the comparison result and determines whether or not the confidentialization level of the information of the item that is a process target has been changed from “∘” or “Δ” to “x” (step 2091).

When the comparison result for the item that is a process target meets the condition of (C7) above, the confidentialization unit 702 determines that the confidentialization level has been changed to “x”. When the comparison result for the item that is a process target meets the condition of (C1), (C4) or (C8) above, the confidentialization unit 702 determines that the confidentialization level has not been changed to “x”.

When the confidentialization level has been changed to “x” (YES in step 2091), the confidentialization unit 702 converts the information of the item that is a process target into data indicating that the information has been confidentialized (step 2092). Then, the confidentialization unit 702 transfers the information after the conversion to the transfer unit 704 (step 2093), and the transfer unit 704 assigns the hospital ID to the received information and transfers the information to the collection storage device 433 (step 2094). The collection storage device 433 overwrites the confidentialized information of the item that is a process target included in the confidentialized personal information 812 in the collection DB 802, with the information received from the transfer unit 704.

When the confidentialization level has not been changed to “x” (NO in step 2091), the confidentialization unit 702 performs the processes in and after step 2095.

When all the items in all the entries having a time and date of updating that is within a collection period have been processed (NO in step 2095), the confidentialization unit 702 checks whether or not there exists an entry of the personal information 611 in a period not overlapping the collection period (step 2101). An entry having a time and date of updating that is later than a collection period corresponds to an entry in a period not overlapping the collection period. When there does not exist an entry in a period not overlapping a collection period (NO in step 2101), the confidentialization unit 702 performs the processes in and after step 2161.

When there exists an entry in a period not overlapping a collection period (YES in step 2101), the time-and-date management unit 703 obtains a confidentialization completion time and date from the time-date table 711 and records the obtained confidentialization completion time and date in the time-date table 712 (step 2102). Then, the time-and-date management unit 703 obtains the last time and date of the collection period from the confidentialization unit 702, records the obtained last time and date as the confidentialization target time and date in the time-date table 712, and sets the process completion flag to “false” (step 2103).

When there does not exist an entry having a time and date of updating that is within a collection period in step 2044 (NO in step 2044), the collection unit 803 instructs the VM 424-1 to establish a connection between the collection DB 802 and the confidentialization unit 702 (step 2104). Then, the collection unit 803 establishes a connection between the collection DB 802 and the confidentialization unit 702 (step 2105), and the VM 424-1 also establishes a connection between the collection DB 802 and the confidentialization unit 702 (step 2106). Then, the VM 424-1 performs the processes in and after step 2102.

Next, the confidentialization unit 702 inquires of the time-and-date management unit 703 about whether or not to perform a confidentialization process (step 2111).

The time-and-date management unit 703 obtains a confidentialization completion time and date and a confidentialization target time and date from the time-date table 712 (step 2112). Then, the time-and-date management unit 703 compares the confidentialization completion time and date and the confidentialization target time and date and transmits, to the confidentialization unit 702, a response indicating whether or not to perform a confidentialization process (step 2113). The time-and-date management unit 703 determines that a confidentialization process is to be performed when the confidentialization target time and date is later than the confidentialization completion time and date and determines that a confidentialization process is not to be performed when the confidentialization target time and date is the same as or earlier than the confidentialization completion time and date.

Next, the confidentialization unit 702 checks the response received from the time-and-date management unit 703 (step 2114), and establishes a connection with the backup storage device 421-1 (step 2115) when a confidentialization process is to be performed (YES in step 2114). When a confidentialization process is not to be performed (NO in step 2114), the confidentialization unit 702 performs the processes in and after step 2161.

Next, the confidentialization unit 702 obtains a confidentialization completion time and date from the time-date table 712 via the time-and-date management unit 703 (step 2121). Then, the confidentialization unit 702 searches the personal information 611 of the backup DB 601 for an entry whose time and date of updating is later than the confidentialization completion time and date (step 2122), and checks whether or not there exists such an entry (step 2123).

When there exists an entry that is later than the confidentialization completion time and date (YES in step 2123), the confidentialization unit 702 obtains that entry from the personal information 611 (step 2124). Then, the confidentialization unit 702 uses the conversion program of the hospital system 411-1 to convert the data format of the obtained entry into the uniform data format (step 2125).

When there does not exist an entry that is earlier than the confidentialization completion time and date (NO in step 2123), the confidentialization unit 702 transmits a process completion report to the time-and-date management unit 703 (step 2126), and performs the processes in and after step 2161. Then, the time-and-date management unit 703 sets the process completion flag to “true” in the time-date table 712 (step 2127).

After performing the process in step 2125, the confidentialization unit 702 uses the personal ID included in the obtained entry to inquire of the identification information assignment unit 425 of the server 423 about a common ID corresponding to the personal ID (step 2131).

The identification information assignment unit 425 searches the ID table 426 for a common ID corresponding to the personal ID (step 2132), and checks whether or not there exists such a common ID (step 2133). When there exists a common ID corresponding to the personal ID (YES in step 2133), the identification information assignment unit 425 reports that common ID to the confidentialization unit 702 (step 2134).

When there does not exist a common ID corresponding to the personal ID (NO in step 2133), the identification information assignment unit 425 assigns a new common ID to that personal ID (step 2135). Then, the identification information assignment unit 425 registers the correspondence relationship between that personal ID and the assigned common ID in the ID table 426 (step 2136) and reports the assigned common ID to the confidentialization unit 702 (step 2134).

Next, the confidentialization unit 702 sets the common ID reported from the identification information assignment unit 425 in the obtained entry (step 2137). Then, the confidentialization unit 702 obtains the confidentialization level information 715 (step 2138) and checks whether or not the symbol is “∘” for each item (step 2141).

When the symbol is “∘” (YES in step 2141), the confidentialization unit 702 transfers the information of that item included in the entry, as it is, to the transfer unit 704 (step 2142). Then, the transfer unit 704 assigns the hospital ID to the received information and transfers the information to the collection storage device 433 of the analysis system 413 (step 2143). When the symbol is not “∘” (NO in step 2141), the confidentialization unit 702 checks whether or not the symbol is “Δ” (step 2144).

When the symbol is “Δ” (YES in step 2144), the confidentialization unit 702 obtains the process table 714 (step 2145), and converts the information of that item included in the entry into simplified information by using the process table 714 (step 2146). Then, the confidentialization unit 702 transfers the information after the conversion to the transfer unit 704 (step 2147), and the transfer unit 704 assigns the hospital ID to the received information and transfers the information to the collection storage device 433 (step 2148).

When the symbol is not “Δ” (NO in step 2144), the confidentialization unit 702 converts the information of that item included in the entry into data indicating that the information has been confidentialized (step 2149). Then, the confidentialization unit 702 transfers the information after the conversion to the transfer unit 704 (step 2150), and the transfer unit 704 assigns the hospital ID to the received information and transfers the information to the collection storage device 433 (step 2151).

The collection storage device 433 stores, in the collection DB 802, the information of the respective items and hospital IDs received from the transfer unit 704, as entries of the confidentialized personal information 812 corresponding to the personal information 611.

Next, the confidentialization unit 702 transmits an update request of the time-date table 712 to the time-and-date management unit 703 (step 2152). In this process, the time-and-date management unit 703 sets, as the confidentialization completion time and date in the time-date table 712, the latest time and date of updating from among the times and dates of updating in entries that have been transferred. When there are a plurality of entries having the latest time and date of updating, the time-and-date management unit 703 sets the number representing the order of an entry that has been transferred, as a same-time sequential number corresponding to the set confidentialization completion time and date.

Next, the confidentialization unit 702 repeats the processes in and after step 2111. When the response indicates that a confidentialization process is not to be performed (NO in step 2114) or when there does not exist an entry that is later than the confidentialization completion time and date (NO in step 2123), the information processing system 401 performs the processes in and after step 2161.

The VM 424-2 through the VM 424-M also perform operations that are similar to those in FIG. 20A through FIG. 20K and generate the confidentialized personal information 812 from the personal information 611 in the backup storage device 421-2 through the backup storage device 421-M.

Next, the analysis application 2001 of the PC 432 obtains the confidentialized personal information 812 from the collection DB 802 of the collection storage device 433 on the basis of a manipulation conducted by the analyst (step 2161) and transmits a collection completion report to the VM 424-1 (step 2162). Then, the analysis application 2001 analyzes the confidentialized personal information 812 on the basis of a manipulation conducted by the analyst (step 2163) and stores an analysis result 2002 in the server 431 (step 2164).

The confidentialization unit 702 of the VM 424-1 that has received the collection completion report switches the transfer destination of the confidentialized personal information from the collection DB 802 to the original collection DB 801 (step 2171). Next, the confidentialization unit 702 switches the process table that it refers to in a confidentialization process from the process table 714 to the original process table 713 (step 2172).

Next, the confidentialization unit 702 switches the confidentialization level information that it refers to in a confidentialization process from the confidentialization level information 715 to the original confidentialization level information 612 (step 2173). Next, the confidentialization unit 702 switches the time-date table that it refers to in a confidentialization process from the time-date table 712 to the original time-date table 711 (step 2174).

Next, the confidentialization unit 702 inquires of the time-and-date management unit 703 about the location at which the confidentialization process in mode M1 was interrupted (step 2175). When the process completion flag is set to “false” in the time-date table 711, the time-and-date management unit 703 transmits, to the confidentialization unit 702, a response including the hospital ID, the confidentialization completion time and date, and the same-time sequential number (step 2176).

“False” as a process completion flag indicates that a confidentialization process in mode M1 was interrupted, and the confidentialization completion time and date and the same-time sequential number represent the location of the interruption in the personal information 611.

The confidentialization unit 702 restarts a confidentialization process in mode M1 for an entry having a time and date of updating that is the same as or later than the confidentialization completion time and date included in the response from among the personal information 611 (step 2177). When there exist a plurality of entries having the same time and date of updating as the confidentialization completion time and date, a confidentialization process is restarted from the entry next to the order specified by the same-time sequential number. When there exists only one entry having the same time and date of updating as the confidentialization completion time and date, a confidentialization process is restarted from the entry having the next time and date of updating. The VM 424-2 through the VM 424-M also perform operations that are similar to those in FIG. 20L and restart a confidentialization process in mode M1.

Note that the information processing apparatus of each hospital may be virtualized by using a container etc. instead of the VM 424-i of each hospital. Virtualization using a container can further increase the speed of a confidentialization process.

In the information processing system 401 illustrated in FIG. 4, the information provision institution may be an institution other than a hospital providing consultation information of a patient. Examples of an information provision institution may include a store that provides customers' purchase information, an educational institution such as a school or a cram school or the like that provide students' grade information, or a financial institution such as a bank that provides customers' balances, records of transactions, etc.

When a store serves as an information provision institution, pieces of customers' purchase information are collected as pieces of personal information, and analysis results representing preferences etc. of the customers are provided to information users such as a restaurant etc. When an educational institution serves as an information provision institution, pieces of students' grade information are collected as pieces of personal information, and analysis results representing tendencies etc. for each subject are provided to information users such as an education material publisher company etc. When a financial institution serves as an information provision institution, pieces of information of customers' balances, transaction records, etc. are collected as pieces of personal information, and analysis results representing a usage of loans etc. are provided to information users such as a loan company etc.

The configurations of the information processing system 201 illustrated in FIG. 2 and the information processing system 401 illustrated in FIG. 4 are just exemplary, and some of the constituents may be omitted or changed in accordance with the purposes or conditions of the information processing systems. For example, in the information processing system 401 illustrated in FIG. 4, when the backup storage device 421-1 can accommodate the backup DBs 601 and the backup DBs 602 of all the hospitals, the other backup storage devices can be omitted. When the VM 424-1 through the VM 424-M can operate in the server 422-1, the servers 422-2 through the servers 422-M can be omitted.

The configurations of the hospital system 411-i illustrated in FIG. 5 and the backup storage device 421-i illustrated in FIG. 6 are just exemplary, and some of the constituents may be omitted or changed in accordance with the purposes or conditions of the information processing system 401. The configurations of the VM 424-i illustrated in FIG. 7 and the collection storage device 433 illustrated in FIG. 8 are just exemplary, and some of the constituents may be omitted or changed in accordance with the purposes or conditions of the information processing system 401.

The flowchart illustrated in FIG. 3 and the operation sequences illustrated in FIG. 19 through FIG. 20L are just exemplary, and some of the processes may be omitted or changed in accordance with the configurations or conditions of the information processing system.

The personal information illustrated in FIG. 9 and FIG. 10, the confidentialization level information illustrated in FIG. 11 and FIG. 12, the process tables illustrated in FIG. 15, and the confidentialized personal information illustrated in FIG. 16 and FIG. 17 are just exemplary, and these pieces of information may be changed in accordance with the content of personal information. The ID table illustrated in FIG. 13 is just exemplary, and an ID table in a different format may be used. For example, information such as a name, a health insurance card ID, etc., which are not national identification numbers, may be used as a personal ID. The time-date table illustrated in FIG. 14 is just exemplary, and a time-date table in a different format may be used. The process of converting a data format illustrated in FIG. 18 is just exemplary, and the data format may be changed in accordance with the items.

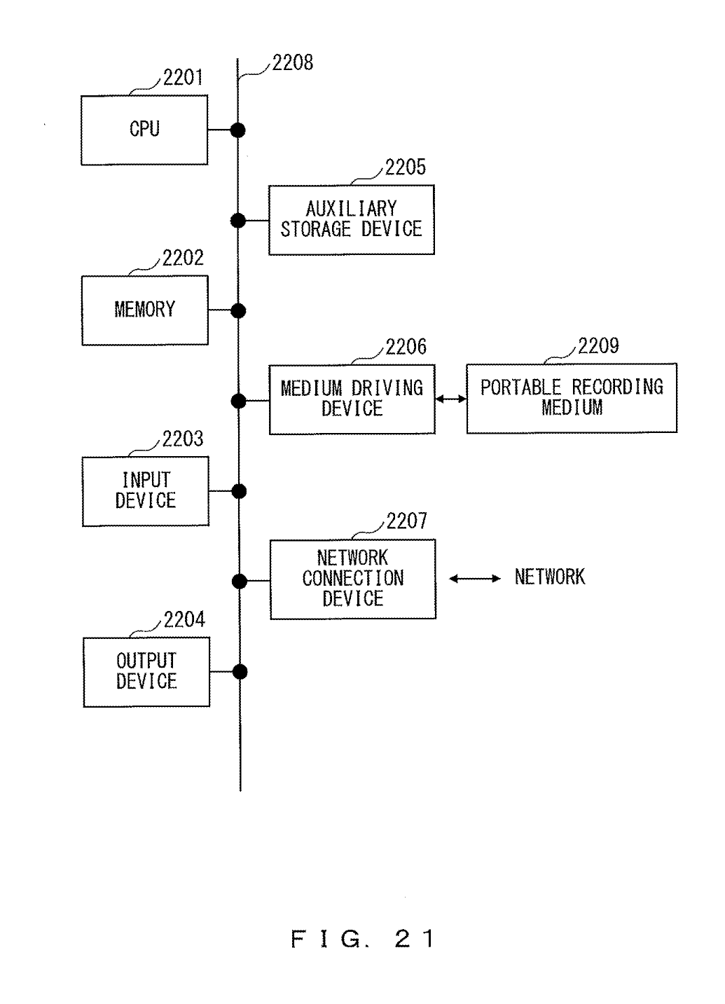

FIG. 21 illustrates a hardware configuration example of an information processing apparatus that is used as the information processing apparatus 212 illustrated in FIG. 2, and as the servers 422-i and 423 and the collection storage device 433 illustrated in FIG. 4. The information processing apparatus illustrated in FIG. 22 includes a Central Processing Unit (CPU) 2201, a memory 2202, an input device 2203, an output device 2204, an auxiliary storage device 2205, a medium driving device 2206, and a network connection device 2207. These constituents are connected to each other via a bus 2208.

The memory 2202 is for example a semiconductor memory such as a Read Only Memory (ROM), a Random Access Memory (RAM), a flash memory, etc., and stores a program and data used for processes. The memory 2202 can be used as the storage unit 224 illustrated in FIG. 2.

The CPU 2201 (processor) executes a program by using for example the memory 2202 so as to operate as the comparison unit 221 and the confidentialization unit 222 illustrated in FIG. 2. The CPU 2201 executes a program by using the memory 2202 so as to operate also as the collection unit 803 and the search unit 804 illustrated in FIG. 8. The CPU 2201 executes a program by using the memory 2202, and thereby makes the VM 424-i illustrated in FIG. 4 operate.

The input device 2203 is for example a keyboard, a pointing device, etc., and is used for inputting instructions or information from the operator or the user. The output device 2204 is for example a display device, a printer, a speaker, etc., and is used for outputting inquiries to the operator or the user or for outputting process results.

The auxiliary storage device 2205 is for example a magnetic disk device, an optical disk device, a magneto-optical disk device, a tape device, etc. The auxiliary storage device 2205 may be a hard disk drive. The information processing apparatus can store a program and data in the auxiliary storage device 2205 beforehand so as to load them onto the memory 2202 and use them. The auxiliary storage device 2205 may be used as the storage unit 224 illustrated in FIG. 2.

The medium driving device 2206 drives a portable recording medium 2209 so as to access information recorded in it. The portable recording medium 2209 is a memory device, a flexible disk, an optical disk, a magneto-optical disk, etc. The portable recording medium 2209 may be a Digital Versatile Disk (DVD), a Compact Disk Read Only Memory (CD-ROM), a Universal Serial Bus (USB) memory, etc. The operator or the user can store a program and data in the portable recording medium 2209 so as to load them onto the memory 2202 and use them.

As described above, a computer-readable recording medium that stores a program and data used for the processes is a physical (non-transitory) recording medium such as the memory 2202, the auxiliary storage device 2205 or the portable recording medium 2209.

The network connection device 2207 is a communication interface circuit that is connected to a communication network such as a LAN, a Wide Area Network (WAN), etc. so as to perform data conversion accompanying communications. The network connection device 2207 may be used as the transfer unit 223 illustrated in FIG. 2. The information processing apparatus can receive a program and data from an external device via the network connection device 2207 and load them onto the memory 2202 so as to use them.

Note that it is not necessary for the information processing apparatuses to include all the constituents illustrated in FIG. 21, and some of the constituents can be omitted in accordance with purposes or conditions. For example, when it is not necessary to input instructions or information from the operator or the user, the input device 2203 can be omitted, and when it is not necessary to output inquiries to the operator or the user or to output process results, the output device 2204 can be omitted. When the portable recording medium 2209 is not used, the medium driving device 2206 can be omitted.

An information processing apparatus that is similar to that illustrated in FIG. 21 can be used as the server 431 and the PC 432 illustrated in FIG. 4 and the PC 501, the PC 502 and the server 503 illustrated in FIG. 5.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. An information processing apparatus comprising:
a memory that stores first confidentialization-level information, which represents a confidentialization level of a first confidentialization process;
a processor that is coupled to the memory and that generates first confidentialized personal information by applying the first confidentialization process to personal information provided from an information provision institution; and
a communication interface circuit that transfers the first confidentialized personal information to a storage device used by an information analysis institution, wherein
the processor compares the first confidentialization-level information and second confidentialization-level information, which represents a confidentialization level requested by the information analysis institution for a second confidentialization process, generates a comparison result, and generates second confidentialized personal information by applying the second confidentialization process to the personal information provided from the information provision institution on the basis of the comparison result, and
the communication interface circuit transfers the second confidentialized personal information to the storage device.

2. The information processing apparatus according to claim 1, wherein
each of the first confidentialization-level information and the second confidentialization-level information specifies a confidentialization operation that is to be applied to each of a plurality of items included in the personal information provided from the information provision institution,
the comparison result indicates whether or not a confidentialization operation specified by the first confidentialization-level information and a confidentialization operation specified by the second confidentialization-level information are the same, for each of the plurality of items,
the processor, for a first item from among the plurality of items, generates confidentialized information of the first item by applying the confidentialization operation specified by the second confidentialization-level information to information of the first item included in the personal information when the confidentialization operation specified by the first confidentialization-level information and the confidentialization operation specified by the second confidentialization-level information are different, and
the communication interface circuit transfers the confidentialized information of the first item to the storage device.

3. The information processing apparatus according to claim 2, wherein
the processor, for a second item from among the plurality of items, refrains from confidentializing information of the second item included in the personal information in the second confidentialization process when the first confidentialization-level information indicates that confidentialization is to be performed and the second confidentialization-level information indicates that confidentialization is not to be performed, and
the communication interface circuit transfers the information of the second item to the storage device.

4. The information processing apparatus according to claim 2, wherein
the processor, for a third item from among the plurality of items, refrains from confidentializing information of the third item included in the personal information in the second confidentialization process when the confidentialization operation specified by the first confidentialization-level information and the confidentialization operation specified by the second confidentialization-level information are the same, and
the communication interface circuit refrains from transferring confidentialized information of the third item to the storage device.

5. The information processing apparatus according to claim 2, wherein
the confidentialization operation specified by the first confidentialization-level information and the confidentialization operation specified by the second confidentialization-level information are a process of simplifying the information of the first item or converting the information of the first item into data indicating that the information of the first item has been confidentialized.

6. An information processing system comprising:
a first storage device that stores personal information provided from an information provision institution;
a second storage device used by an information analysis institution; and
an information processing apparatus that generates first confidentialized personal information by applying a first confidentialization process to the personal information stored in the first storage device, transfers the first confidentialized personal information to the second storage device, generates second confidentialized personal information by applying a second confidentialization process to the personal information stored in the first storage device on the basis of a result of a comparison between first confidentialization-level information, which represents a confidentialization level of the first confidentialization process, and second confidentialization-level information, which represents a confidentialization level requested by the information analysis institution for the second confidentialization process, and transfers the second confidentialized personal information to the second storage device.

7. The information processing system according to claim 6, wherein
each of the first confidentialization-level information and the second confidentialization-level information specifies a confidentialization operation to be applied to each of a plurality of items included in the personal information stored in the first storage device,
the comparison result indicates whether or not a confidentialization operation specified by the first confidentialization-level information and a confidentialization operation specified by the second confidentialization-level information are the same, for each of the plurality of items, and
the information processing apparatus, for a first item from among the plurality of items, generates confidentialized information of the first item by applying the confidentialization operation specified by the second confidentialization-level information to information of the first item included in the personal information and transfers the confidentialized information of the first item to the second storage device, when the confidentialization operation specified by the first confidentialization-level information and the confidentialization operation specified by the second confidentialization-level information are different.

8. The information processing system according to claim 7, wherein
the information processing apparatus, for a second item from among the plurality of items, refrains from confidentializing information of the second item included in the personal information in the second confidentialization process and transfers the information of the second item to the second storage device, when the first confidentialization-level information indicates that confidentialization is to be performed and the second confidentialization-level information indicates that confidentialization is not to be performed.

9. The information processing system according to claim 7, wherein
the information processing apparatus, for a third item from among the plurality of items, refrains from confidentializing information of the third item included in the personal information in the second confidentialization process and refrains from transferring confidentialized information of the third item to the second storage device, when the confidentialization operation specified by the first confidentialization-level information and the confidentialization operation specified by the second confidentialization-level information are the same, and
the second storage device uses confidentialized information of the third item included in the first confidentialized personal information as confidentialized information of the third item in the second confidentialized personal information.

10. The information processing system according to claim 7, wherein
the confidentialization operation specified by the first confidentialization-level information and the confidentialization operation specified by the second confidentialization-level information are a process of simplifying the information of the first item or converting the information of the first item into data indicating that the information of the first item has been confidentialized.

11. A non-transitory computer-readable recording medium having stored therein a program that causes a computer to execute a process comprising:
generating first confidentialized personal information by applying a first confidentialization process to personal information provided from an information provision institution;
transferring the first confidentialized personal information to a storage device used by an information analysis institution;
comparing first confidentialization-level information, which represents a confidentialization level of the first confidentialization process, and second confidentialization-level information, which represents a confidentialization level requested by the information analysis institution for a second confidentialization process, and generating a comparison result;
generating second confidentialized personal information by applying the second confidentialization process to the personal information provided from the information provision institution on the basis of the comparison result, and
transferring the second confidentialized personal information to the storage device.

12. The non-transitory computer-readable recording medium according to claim 11, wherein
each of the first confidentialization-level information and the second confidentialization-level information specifies a confidentialization operation that is to be applied to each of a plurality of items included in the personal information provided from the information provision institution,
the comparison result indicates whether or not a confidentialization operation specified by the first confidentialization-level information and a confidentialization operation specified by the second confidentialization-level information are the same, for each of the plurality of items,
the generating the second confidentialized personal information generates, for a first item from among the plurality of items, confidentializated information of the first item by applying the confidentialization operation specified by the second confidentialization-level information to information of the first item included in the personal information when the confidentialization operation specified by the first confidentialization-level information and the confidentialization operation specified by the second confidentialization-level information are different, and
transferring the second confidentialized personal information transfers the confidentialized information of the first item to the storage device.

13. An information processing method comprising:
generating, by a processor, first confidentialized personal information by applying a first confidentialization process to personal information provided from an information provision institution;
transferring the first confidentialized personal information to a storage device used by an information analysis institution;
comparing, by the processor, first confidentialization-level information, which represents a confidentialization level of the first confidentialization process, and second confidentialization-level information, which represents a confidentialization level requested by the information analysis institution for a second confidentialization process, and generating a comparison result;
generating, by the processor, second confidentialized personal information by applying the second confidentialization process to the personal information provided from the information provision institution on the basis of the comparison result; and
transferring the second confidentialized personal information to the storage device.

14. The information processing method according to claim 13, wherein
each of the first confidentialization-level information and the second confidentialization-level information specifies a confidentialization operation that is to be applied to each of a plurality of items included in the personal information provided from the information provision institution,
the comparison result indicates whether or not a confidentialization operation specified by the first confidentialization-level information and a confidentialization operation specified by the second confidentialization-level information are the same, for each of the plurality of items,
the generating the second confidentialized personal information generates, for a first item from among the plurality of items, confidentialized information of the first item by applying the confidentialization operation specified by the second confidentialization-level information to information of the first item included in the personal information when the confidentialization operation specified by the first confidentialization-level information and the confidentialization operation specified by the second confidentialization-level information are different, and
transferring the second confidentialized personal information transfers the confidentialized information of the first item to the storage device.
